(19) United States
(12) Patent Application Publication
(10) Pub. No.: US 2026/0283966 A1
(43) Pub. Date: Sep. 24, 2026

(54) PROTEIN PARTICLES AND METHODS OF MAKING AND USING THE SAME

(71) Applicants: Dairy Management Inc., Rosemont, IL (US); Cornell University, Ithaca, NY (US)

(72) Inventors: Hari Meletharayil, Palatine, IL (US); Emil S. Nashed, North Haledon, NJ (US); Rohit Kapoor, Elk Grove Village, IL (US); Hongmin Dong, Ithaca, NY (US); Younas Dadmohammadi, Ithaca, NY (US); Alireza Abbaspourrad, Ithaca, NY (US); Lixin Yang, Ithaca, NY (US); Tiantian Lin, Ithaca, NY (US)

(21) Appl. No.: 19/473,544

(22) PCT Filed: Apr. 12, 2024
(86) PCT No.: PCT/US2024/024231

§ 371 Date: Oct. 8, 2025

Related U.S. Application Data
(60) Provisional application No. 63/495,883, filed on Apr. 13, 2023.

(51) Int. Cl. A61K 9/50; A61K 9/51; A61K 31/198; A61K 31/355; A61K 31/405

(52) U.S. Cl. CPC A61K 9/5052; A61K 9/5192; A61K 31/198; A61K 31/355; A61K 31/405

(57) ABSTRACT

Described herein are particles including a protein and optionally an active agent such as a particle comprising whey protein hydrolysate and optionally tryptophan. Also described herein are methods of making and using particles that include a protein and optionally an active agent.

### STATEMENT REGARDING ELECTRONIC FILING OF A SEQUENCE LISTING

A Sequence Listing in XML format, entitled 1213-9WO_ST26.xml, 4,495 bytes in size, generated on Apr. 2, 2024, and filed herewith, is hereby incorporated by reference in its entirety for its disclosures.

### FIELD

The present invention relates to particles including a protein and optionally an active agent and to methods of making and using such particles.

### BACKGROUND

L-Tryptophan (Trp) is a nonpolar aromatic essential amino acid and can be obtained from dietary protein. It is a precursor to key biomolecules such as serotonin, melatonin, tryptamine, niacin, quinolinic acid, and kynurenic acid-nicotinamide adenine dinucleotide. Therefore, Trp plays an important role in regulating neurobehavioral processes such as appetite, mood, sleep, cognition, sense of pain, and behavior. Supplementation with this amino acid has been demonstrated to be effective in the medical treatment of several diseases, including depression, sleep disorders, cognitive disorders, anxiety, and neurodegenerative disease. In addition, Trp demonstrated angiotensin-converting enzyme (ACE) inhibition, antioxidant, antidiabetic, and satiating effects (Nongonierma & FitzGerald, 2015). Because of these properties, Trp is considered an essential dietary component. The application of tryptophan, however, is limited due to its notable bitter taste, which is attributed to aromatic and hydrophobic residues. Among the free amino acids, Trp has the lowest bitter taste threshold (BTT: 4 mmol/L) (Di Pizio & Nicoli, 2020 Molecules (Basel, Switzerland), 25 (20), doi.org/10.3390/molecules25204623). The bitter taste of Trp can lead to a reduction in consumption of Trp-containing nutritional supplements and limit its application in food ingredients.

### SUMMARY

A first aspect of the present invention is directed to a method of preparing a particle of the present invention, the method comprising: providing a protein in a composition (e.g., dissolved and/or suspended in an aqueous composition); adjusting the pH of the composition to a basic pH, then adjusting the pH of the composition to an acidic pH; and optionally, then heating the composition, thereby forming the particle that comprises the protein.

A second aspect of the present invention is directed to a method of preparing a particle of the present invention, the method comprising: combining a protein and an active agent to form a mixture; adjusting the pH of the mixture to a basic pH, then adjusting the pH of the mixture to an acidic pH; and optionally, then heating the mixture, thereby forming the particle that comprises the protein and the active agent.

A further aspect of the present invention is directed to a particle comprising: a protein; and optionally an active agent. In some embodiments, when the active agent is present in the particle, the active agent is present within the protein (e.g., within the tertiary structure of the protein). In some embodiments, when the active agent is present in the particle, the active agent is nonspecifically bound (e.g., via hydrophobic interaction, electrostatic interaction, and/or hydrogen bonding, etc.) to the protein.

An additional aspect of the present invention is directed to a plurality of particles of the present invention.

A further aspect of the present invention is directed to a composition comprising a carrier (e.g., water and/or an oil) and a particle of the present invention (e.g., a particle prepared according to a method of the present invention). In some embodiments, the composition is a dispersion.

A further aspect of the present invention is directed to an article comprising a particle of the present invention and/or a composition of the present invention. In some embodiments, the article is a food product (e.g., infant formula, a dairy product, etc.), nutritional supplement, therapeutic drink, and/or cosmetic.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

FIG. 1 is a graph showing the particle size distribution of WPI-Trp nanoparticles formed by pH shifting from either 11 to 6 or 11 to 7 followed by heat treatment at 70° C. for 60 minutes.

FIG. 2 is a graph showing the effect of pH shifting alone and pH shifting followed by heat treatment on the particle size of WPI-Trp mixtures. Different letters indicate a significant difference (p<0.05).

FIG. 3 is a graph showing the intrinsic fluorescence spectra of WPI-Trp mixtures that were subjected to pH shifting alone and pH shifting followed by heat treatment.

FIG. 4 is a schematic of a proposed mechanism of aggregation-induced fluorescence emission of Trp.

FIG. 5 is a graph showing the particle size and polydispersity index (PDI) of suspensions including native whey protein isolate (WPI) particles, WPI-Trp nanoparticles with pH shifting from 11 to 6 only, and WPI-Trp nanoparticles with pH shifting from 11 to 6 followed by heat treatment at either 50° C., 60° C., 70° C., or 80° C. for 60 minutes.

FIG. 6 is a graph showing the particle size of pH-shifted (pH 11 to pH 6) WPI-Trp mixtures heated at various temperatures (50/60/70/80° C.).

FIG. 7 is a graph of the intrinsic fluorescence spectra of pH-shifted (pH 11 to pH 6) WPI-Trp mixtures heated at various temperatures (50/60/70/80° C.).

FIG. 8 shows graphs of the particle size distribution for WPI-Trp samples that underwent pH shifting from pH 11 to pH 6 and heat treatment at 70° C. for a period of between 0 and 60 minutes based on intensity percentage or volume percentage.

FIG. 9 shows graphs of particle size distribution for WPI samples that underwent pH shifting from pH 11 to pH 6 and heat treatment at 70° C. for a period of between 0 and 60 minutes based on intensity percentage or volume percentage.

FIG. 10 shows graphs of particle size distribution for Trp samples that underwent pH shifting from pH 11 to pH 6 and heat treatment at 70° C. for a period of between 0 and 60 minutes based on intensity percentage or volume percentage.

FIG. 11 shows graphs of particle size distribution for WPI-Trp samples at pH 6 that did not undergo pH shifting and underwent heat treatment at 70° C. for a period of between 0 and 60 minutes based on intensity percentage or volume percentage.

FIG. 12 is a graph showing the turbidity of samples of WPI-Trp, WPI, and Trp that underwent pH shifting from pH 11 to pH 6 and heat treatment at 70° C. for a period of between 0 and 60 minutes.

FIG. 13 is a graph showing the turbidity of samples of WPI-Trp, WPI, and Trp at pH 6 that did not undergo pH shifting and underwent heat treatment at 70° C. for a period of between 0 and 60 minutes.

FIG. 14 is a graph showing particle size and polydispersity index (PDI) for both WPI-Trp nanoparticles and WPI nanoparticles after pH shifting from 11 to 6 followed by heat treatment at 70° C. for either 0, 10, 20, 30, 40, 50, or 60 minutes.

FIG. 15 shows graphs of the intrinsic fluorescence spectra of WPI-Trp (FIG. 15, Panel A), WPI (FIG. 15, Panel B), and Trp (FIG. 15, Panel C) samples that underwent pH shifting from pH 11 to pH 6 and heat treatment at 70° C. for a period of between 0 and 60 minutes.

FIG. 16 is a graph showing the particle size of WPI-Trp mixture at pH 6, WPI-Trp mixture with heating only, WPI-Trp mixture with pH shifting from pH 11 to pH 6 only, and WPI-Trp NPs with pH shifting from pH 11 to pH 6 combined with heating. Different letters indicate a significant difference (p<0.05).

FIG. 17 is a graph of the intrinsic fluorescence spectra of WPI-Trp mixture at pH 6, WPI-Trp mixture with heating only, WPI-Trp mixture with pH shifting from pH 11 to pH 6 only, and WPI-Trp NPs with pH shifting from pH 11 to pH 6 combined with heating.

FIG. 18 is a graph showing the particle size distribution of WPI-Trp nanoparticles formed with pH shifting from 11 to 6 followed by heat treatment at 70° C. for 20 minutes.

FIG. 19 is a graph showing the particle size distribution of WPI-Trp nanoparticles formed without pH shifting from 11 to 6 followed by heat treatment at 70° C. for 20 minutes.

FIG. 20 shows SEM images of WPI-Trp mixtures under different treatments before and after freeze-drying. FIG. 20, Panel A shows freeze-dried WPI-Trp samples at pH 6; FIG. 20, Panel B shows freeze-dried WPI-Trp samples after heat treatment (70° C., 20 min) at pH 6; FIG. 20, Panel C shows freeze-dried WPI-Trp samples after pH shifting from 11 to 6; FIG. 20, Panel D shows freeze-dried WPI-Trp samples obtained by pH shifting from 11 to 6 followed by heat treatment (70° C., 20 min); FIG. 20, Panel E shows WPI-Trp samples after heat treatment (70° C., 20 min) at pH 6 before freeze-drying; and FIG. 20, Panel F shows WPI-Trp NPs before freeze-drying: pH shifting (from 11 to 6) followed by heat treatment (70° C., 20 min).

FIG. 21 is a graph showing the surface hydrophobicity (H₀) of WPI-Trp mixtures with different treatments. Different letters indicate a significant difference (p<0.05).

FIG. 22 is a graph showing the surface hydrophobicity (H₀) of WPI with different treatments. Different letters indicate a significant difference (p<0.05).

FIG. 23 is a graph showing the free sulfhydryl (SH) group content of WPI-Trp mixtures with different treatments. Different letters indicate a significant difference (p<0.05).

FIG. 24 is a graph showing the free sulfhydryl (SH) group content of WPI with different treatments. Different letters indicate a significant difference (p<0.05).

FIG. 25 is a graph of the circular dichroism (CD) spectra of WPI-Trp with different treatments.

FIG. 26 shows the circular dichroism (CD) spectra of WPI with different treatments.

FIG. 27 shows the FTIR spectra of WPI-Trp NPs, native WPI, and native Trp.

FIG. 28 is a graph of the DPPH radical scavenging capacity of WPI-Trp at pH 6, with heating at 70° C. for 20 minutes only, with pH shifting from pH 11 to pH 6 only, and with both pH shifting from pH 11 to pH 6 and heating at 70° C. for 20 minutes.

FIG. 29 is a graph of the DPPH radical scavenging capacity of WPI native at pH 6, with heating at 70° C. for 20 minutes only, with pH shifting from pH 11 to pH 6 only, and with both pH shifting from pH 11 to pH 6 and heating at 70° C. for 20 minutes.

FIG. 30 is a graph of the DPPH radical scavenging capacity of Trp native at pH 6, with heating at 70° C. for 20 minutes only, with pH shifting from pH 11 to pH 6 only, and with both pH shifting from pH 11 to pH 6 and heating at 70° C. for 20 minutes.

FIG. 31 is a graph of the ABTS radical scavenging capacity of WPI-Trp at 0.05 mg ml⁻¹ at pH 6, with heating at 70° C. for 20 minutes only, with pH shifting from pH 11 to pH 6 only, and with both pH shifting from pH 11 to pH 6 and heating at 70° C. for 20 minutes. Different letters indicate a significant difference (p<0.05).

FIG. 32 is a graph of the ABTS radical scavenging capacity of WPI at 0.05 mg ml⁻¹ native at pH 6, with heating at 70° C. for 20 minutes only, with pH shifting from pH 11 to pH 6 only, and in with both pH shifting from pH 11 to pH 6 and heating at 70° C. for 20 minutes. Different letters indicate a significant difference (p<0.05).

FIG. 33 is a graph of the ABTS radical scavenging capacity of Trp at 0.05 mg ml⁻¹ native at pH 6, with heating at 70° C. for 20 minutes only, with pH shifting from pH 11 to pH 6 only, and with both pH shifting from pH 11 to pH 6 and heating at 70° C. for 20 minutes. Different letters indicate a significant difference (p<0.05).

FIG. 34 shows the molecular docking complexes of α-LA-Trp (FIG. 34, Panel A), β-LG-Trp (FIG. 34, Panel B), and BSA-Trp (FIG. 34, Panel C), including details of binding sites of α-LA-Trp, β-LG-Trp, and BSA-Trp. Green dashed lines show a hydrogen bond, and amino acid residues colored as red indicate hydrophobic interaction with the Trp.

FIG. 35 is a schematic of a proposed mechanism for the formation of WPI-Trp nanoparticles.

FIG. 36 is a flow diagram of an exemplary process for preparing whey protein isolate-tryptophan nanoparticles using ultrafiltration or nanofiltration.

### DETAILED DESCRIPTION OF EXAMPLE EMBODIMENTS

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting to the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B, and C, it is specifically intended that any of A, B, or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also, as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancing,” “improve” and “improving” (and grammatical variations thereof) describe an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% such as compared to another measurable property or quantity (e.g., a control value). In some embodiments, the reduction can result in no or essentially no (i.e., an insignificant amount, e.g., less than about 10% or even 5%) detectable activity or amount.

A “portion” or “fragment” of a nucleotide sequence or polypeptide (including a domain) will be understood to mean a nucleotide sequence or polypeptide of reduced length (e.g., reduced by 1, 2, 3, 4, 5, 6, 7, 8, 9, 10, 11, 12, 13, 14, 15, 16, 17, 18, 19, 20 or more residue(s) (e.g., nucleotide(s) or peptide(s)) relative to a reference nucleotide sequence or polypeptide, respectively, and comprising, consisting essentially of and/or consisting of a nucleotide sequence or polypeptide of contiguous residues, respectively, identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference nucleotide sequence or polypeptide.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence as compared to a reference polypeptide.

As used herein, the phrase “substantially identical,” or “substantial identity” in the context of two nucleic acid molecules, nucleotide sequences or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, or more nucleotides in length, and any range therein, up to the full length of the sequence. In some embodiments, the nucleotide sequences can be substantially identical over at least about 20 nucleotides (e.g., about 20, 21, 22, 23, 24, 25, 26, 27, 28, 29, 30, 31, 32, 33, 34, 35, 36, 37, 38, 39, 40 nucleotides). In some embodiments, a substantially identical nucleotide or protein sequence performs substantially the same function as the nucleotide (or encoded protein sequence) to which it is substantially identical.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Provided according to embodiments of the present invention are particles that comprise a protein and optionally an active agent. When a particle of the present invention comprises a protein and an active agent, the active agent may be associated with the protein. In some embodiments, a particle of the present invention comprises a protein and an active agent that is associated with the protein. In some embodiments, a particle of the present invention comprises a protein and is devoid of an active agent. In some embodiments, when an active agent is present in a particle of the present invention, an active agent is present within a protein present in a particle of the present invention and/or an active agent is present on a surface of a protein present in a particle of the present invention. In some embodiments, the active agent is present within the tertiary structure of the protein. In some embodiments, the active agent is nonspecifically bound, such as via a hydrophobic interaction, electrostatic interaction, hydrogen bonding, and/or the like, to the protein. One or more (e.g., 1, 2, 3, 4, 5, 6, 7, 8, 9, 10, 15, 20, 25, 30, or more) protein molecule(s) (e.g., protein monomer(s)) may be present in a particle of the present invention. In some embodiments, one or more (e.g., 1, 2, 3, 4, 5, 6, 7, 8, 9, 10, 15, 20, 25, 30, or more) active agent(s) are present between two or more protein molecules that are associated with one another (e.g., via a non-specific interaction).

A particle of the present invention may comprise one or more (e.g., 1, 5, 10, 20, 30, 40, 50 or more) active agent(s), which may be the same or different from each other, and/or one or more (e.g., 1, 5, 10, 20, 30, 40, 50 or more) protein molecule(s), which may be the same or different from each other. In some embodiments, a particle of the present invention comprises an active agent that is within an area of the tertiary structure (e.g., within a tertiary fold) of the protein. In some embodiments, the active agent is present within an area of the tertiary structure (e.g., within a tertiary fold) of the protein that comprises at least one nonspecific hydrophobic interaction between two or more amino acid residues. In some embodiments, the active agent is present in a hydrophobic pocket of the protein. In some embodiments, the active agent is within the protein core of the protein. In some embodiments, the active agent is within a folded region of the protein that optionally has zero solvent accessibility. In some embodiments, when a particle of the present invention comprises a plurality of active agents (where the active agents in the plurality of active agents may be the same or different from each other), at least one active agent of the plurality of active agents is present within the protein and one or more of the active agent(s) of the plurality of active agents may be present on a surface of the protein.

Exemplary proteins of the present invention include, but are not limited to, dairy proteins (e.g., milk proteins), plant proteins, and/or animal (e.g., meat) proteins. A “dairy protein,” “milk protein,” “plant protein,” “animal protein,” and “meat protein,” as used herein refer to a protein that is found naturally in a dairy product, milk, plant, animal, and meat, respectively, and/or that is derived from such a naturally occurring protein to have an amino acid sequence having at least 70% sequence identity to the naturally occurring protein's amino acid sequence. For example, in some embodiments, a dairy, milk, plant, animal, or meat protein is naturally found in a dairy product, milk, plant, animal, or meat, respectively, and/or the protein is isolated from the dairy product, milk, plant, animal, or meat, respectively, or the protein is synthetically prepared to have an amino acid sequence having at least 70% sequence identity to the naturally occurring protein's amino acid sequence. In some embodiments, the protein is a milk protein such as, but not limited to, α-lactalbumin, β-lactoglobulin, and/or lactoferrin. In some embodiments, whey protein isolate (WPI), which comprises α-lactalbumin and β-lactoglobulin and may comprise lactoferrin, is used to prepare a particle of the present invention. In some embodiments, a whey protein isolate (WPI) comprises about 50%, 55%, 60%, 65%, 70%, 80% or 90% β-lactoglobulin and/or about 20%, 25%, 30%, 35%, 40%, 50%, 60%, 70%, 80% or 90% α-lactalbumin. In some embodiments, a particle of the present invention may comprise whey protein isolate (WPI) and is devoid of an active agent. In some embodiments, a particle of the present invention comprises one or more of α-lactalbumin and one or more of β-lactoglobulin.

A protein that is used to prepare a particle of the present invention may have a molten globule state and/or have a bilobal structure. The protein may comprise two or more (e.g., 2, 3, 4, 5, or more) domains and/or the protein, at a pH of about 5 to about 9, may comprise one or more (e.g., 1, 2, 3, 4, or more) intramolecular disulfide bonds. In some embodiments, the protein comprises at least two domains and the protein comprises at least one disulfide bridge that connects two domains of the protein. In some embodiments, the protein is monomeric. In some embodiments, the protein has about 100 amino acids to about 200, 300, 400, or 500 amino acids and/or a molecular weight of about 10 kDa to about 20, 30, 40, or 50 kDa. The protein may have about 100, 150, 200, 250, 300, 350, 400, 450, or 500 amino acids. In some embodiments, the protein has a molecular weight of about 10, 15, 20, 25, 30, 35, 40, 45, or 50 kDa. The protein may have an isoelectric point (pI) of about 4, 4.1, 4.2, 4.3, or 4.4 to about 4.5, 4.6, 4.7, 4.8, 4.9, 5, 5.1, 5.2, 5.3 5.4, or 5.5. In some embodiments, the protein has a pI of about 4.2 to about 4.5 or 5.2. The protein may have a structure that comprises α-helices in an amount of about 10% or 15% to about 20%, 25%, or 30% of the total tertiary and/or secondary structure (optionally calculated by the percentage of the number of amino acids present in an α-helix to the total number of amino acids in the protein), β-sheets in an amount of about 1% or 5% to about 10%, 15%, 20%, or 25% of the total tertiary and/or secondary structure (optionally calculated by the percentage of the number of amino acids present in a β-sheet to the total number of amino acids in the protein), β-turns in an amount of about 1% or 5% to about 10%, 15%, 20%, or 25% of the total tertiary and/or secondary structure (optionally calculated by the percentage of the number of amino acids present in a β-turn to the total number of amino acids in the protein), and/or an unordered tertiary and/or secondary structure in an amount about 30%, 35%, 40%, 45, 50%, 55%, or 60% to about 65%, 70%, or 75% of the total tertiary and/or secondary structure (optionally calculated by the percentage of the number of amino acids present in an unordered tertiary and/or secondary structure to the total number of amino acids in the protein).

A particle of the present invention may comprise a plurality of proteins. In some embodiments, a particle of the present invention comprises about 5, 10, or 20 to about 25, 30, 40, or 50 protein molecules (e.g., protein monomers). In some embodiments, a particle of the present invention comprises about 5, 10, 15, 20, 25, 30, 35, 40, 45, or 50 protein molecules (e.g., protein monomers). A particle of the present invention may comprise a protein in a total amount (e.g., one or more protein molecules) of about 75%, 80%, 85%, to about 90%, 95%, 99%, or 100% by weight of the particle. In some embodiments, a protein may be present in the particle in an amount of about 100% by weight of the particle. In some embodiments, an active agent may be present in the particle in a total amount (e.g., one or more active agents) of about 0%, 0.1%, 0.5%, 1% or 5% to about 10%, 15%, 20%, or 25% by weight of the particle. In some embodiments, the active agent may be present in the particle in an amount of about 10%, 11%, 12%, 13%, or 14% to about 15%, 16%, 17%, 18%, 19%, or 20% by weight of the particle. In some embodiments, a particle of the present invention may comprise a protein in a total amount (e.g., one or more protein molecules) of about 75%, 80%, 85%, to about 90%, 95%, 99%, or 100% by weight of the particle and an active agent in a total amount (e.g., one or more active agents) of about 0%, 0.1%, 0.5%, 1% or 5% to about 10%, 15%, 20%, or 25% by weight of the particle.

Further exemplary proteins that may be present in a particle of the present invention include, but are not limited to, whey protein isolate, α-lactalbumin, lysozyme, cytochrome c, apomyoglobin, staphylococcal nuclease, β-lactoglobulin, lactoferrin, and any combination thereof. In some embodiments, a protein that may be present in a particle of the present invention is whey protein isolate. A protein of the present invention may be from any source (e.g., plant, animal, etc.). In some embodiments, the protein is obtained and/or derived from an animal source such as a mammal (e.g., a bovine or human). In some embodiments, a particle of the present invention comprises about 5, 10, 15, 20, 25, 30, 35, 40, 45, or 50 whey protein isolate molecules. In some embodiments, a protein present in a particle of the present invention has an amino acid sequence having about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to one or more of SEQ ID NOs: 1-3. In some embodiments, a protein present in a particle of the present invention has an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more sequence identity to one or more of SEQ ID NOs: 1-3. In some embodiments, a protein present in a particle of the present invention has an amino acid sequence having about 100% sequence identity to one or more of SEQ ID NOs: 1-3.

An active agent used to prepare a particle of the present invention may be an organic compound such as, but not limited to, an amino acid. In some embodiments, the active agent has a molecular weight of about 70, 100, 150, or 200 g/mol to about 250, 300, 400, or 500 g/mol. The active agent may have a solubility in water at 25° C. of about 15 mg/mL or less such as about 15, 14, 13, 12, 11, 10, 9, 8, 7, 6, or 5 mg/L, or less. In some embodiments, the active agent may have a solubility in water at 25° C. of about 5, 6, 7, 8, 9, or 10 mg/mL to about 11, 12, 13, 14, or 15 mg/L. In some embodiments, the active agent has a pKa of about 1.5, 2, 2.5, 2.6, 2.7, or 2.8 to about 2.9, 3, 3.2, or 3.5 and/or a pI of about 5, 5.5, 5.6, 5.7, or 5.8 to about 5.9, 6, 6.1, 6.2, 6.3, 6.4, or 6.5. In some embodiments, the active agent has a solubility in water of about 10 mg/mL at 25° C. or less and/or has a pKa of about 2.7 or 2.8 to about 2.9 or 3 and/or a pI of about 5.7 or 5.8 to about 5.9, 6, or 6.1. Exemplary active agents include, but are not limited to, amino acids (e.g., tryptophan, leucine, phenylalanine, cysteine, and/or tyrosine), vitamin E, and any combination thereof. In some embodiments, the active agent present in a particle of the present invention is tryptophan.

A particle of the present invention may have a size (e.g., a diameter) in at least one dimension of about 50, 75, or 100 nm to about 125, 150, 200, 250, 300, 350, 400, 450, 500, 550, 600, 650, 700, 750, or 800 nm, optionally as measured using microscopy (e.g., scanning electron microscopy (SEM) and/or transmission electron microscopy (TEM) and/or dynamic light scattering (DLS). In some embodiments, the particle has a size (e.g., a diameter) in at least one dimension of about 50, 75, 100, 125, 150, 200, 250, 300, 350, 400, 450, 500, 550, 600, 650, 700, 750, or 800 nm. In some embodiments, the particle has a size (e.g., diameter) in at least one dimension of about 125 nm. In some embodiments, the particle has a size (e.g., diameter) in at least one dimension of about 110 nm. In some embodiments, the particle is a nanoparticle. In some embodiments, a plurality of particles of the present invention, particles prepared according to a method of the present invention, and/or particles present in a composition of the present invention have a Dv(50) of about 50, 75, or 100 nm to about 125, 150, or 200 nm, optionally as measured using microscopy (e.g., SEM and/or TEM) and/or DLS. In some embodiments, a plurality of particles of the present invention have a Dv(50) of about 50, 75, 100, 125, 150, or 200 nm, optionally as measured using microscopy (e.g., SEM and/or TEM) and/or DLS. In some embodiments, a plurality of particles of the present invention have a polydispersity index (PDI) of less than about 0.5, optionally of less than about 0.3. In some embodiments, a plurality of particles of the present invention have a polydispersity index (PDI) of about 0.2.

In some embodiments, a particle of the present invention has a spherical structure. In some embodiments, a particle of the present invention has a rod-like structure. In some embodiments, a particle of the present invention has a cube-like structure. In some embodiments, a particle of the present invention has a loose matrix structure. In some embodiments, each particle of a plurality of particles of the present invention, when present in a composition (e.g., a liquid such as water, a buffer, a milk (e.g., skim milk), and/or an acid whey beverage), have the same or a different structure selected from a spherical structure, a rod-like structure, a cube-like structure, and/or a loose matrix structure. In some embodiments, a particle of the present invention may have a structure that comprises α-helices in an amount of about 10% or 15% to about 20%, 25%, or 30% of the total tertiary and/or secondary structure (optionally calculated by the percentage of the number of amino acids present in an α-helix to the total number of amino acids in the protein), β-sheets in an amount of about 1% or 5% to about 10%, 15%, 20%, 25%, 30%, or 35% of the total tertiary and/or secondary structure (optionally calculated by the percentage of the number of amino acids present in a β-sheet to the total number of amino acids in the protein), β-turns in an amount of about 1% or 5% to about 10%, 15%, 20%, or 25% of the total tertiary and/or secondary structure (optionally calculated by the percentage of the number of amino acids present in a β-turn to the total number of amino acids in the protein), and/or an unordered tertiary and/or secondary structure in an amount about 30%, 35%, 40%, 45, 50%, 55%, or 60% to about 65%, 70%, or 75% of the total tertiary and/or secondary structure (optionally calculated by the percentage of the number of amino acids present in an unordered tertiary and/or secondary structure to the total number of amino acids in the protein). In some embodiments, upon exposure to a temperature of about 70° C. for about 20 minutes, the amount of one or more of α-helices, β-sheets, β-turns, and unordered tertiary and/or secondary structure present in a particle of the present invention remain within about #5%, 10%, 15%, 20%, 25%, 30%, or 35% of the amount of α-helices, β-sheets, β-turns, and unordered tertiary and/or secondary structure, respectively, present in the particle prior to the exposure (e.g., the amount of α-helices, β-sheets, β-turns, and unordered tertiary and/or secondary structure each present in the particle at initial formation of the particle and/or immediately prior to the exposure).

In some embodiments, a particle of the present invention may have a free sulfhydryl (SH) group content of about 5, 6, 7, 8, 9, or 10 μmol SH/g to about 11, 12, 13, 14, or 15 μmol SH/g, optionally as measured using Ellman's reagent (5,5′-dithiobis-(2-nitrobenzoic acid) or DTNB) and UV-Vis spectroscopy. In some embodiments, a particle of the present invention may have a free sulfhydryl (SH) group content of about 5, 6, 7, 8, 9, 10, 11, 12, 13, 14, or 15 μmol SH/g, optionally as measured using Ellman's reagent (5,5′-dithiobis-(2-nitrobenzoic acid) or DTNB) and UV-Vis spectroscopy. In some embodiments, a particle of the present invention may have a surface hydrophobicity of about 4×10⁷ to about 7×10⁷, optionally as measured using 8-anilino-1-naphthalene sulfonate (ANS) as a fluorescence probe. In some embodiments, a particle of the present invention comprising a protein and an active agent may have an increased intrinsic fluorescence intensity compared to the intrinsic fluorescence intensity of the protein and/or active agent alone (e.g., the protein and/or active agent not present in a particle of the present invention).

In some embodiments, a protein and/or an active agent that are used to prepare a particle of the present invention dissolve in water at a temperature of about 25° C. and a pH of about 11. In some embodiments, the protein and/or active agent dissolve in water at a temperature of about 25° C. and a pH of about 11 in an amount of about 25, 30, 40, or 45 mg/mL to about 50, 100, 150, 200, 250, 300, 350, or 400 mg/L The protein and/or active agent may have a negative charge in water at a pH of about 11.

A particle of the present invention comprising a protein and optionally an active agent may have improved (e.g., increased) storage, stability, activity, and/or function compared to the storage, stability, activity, and/or function of the protein alone (e.g., the protein not present in a particle of the present invention and not in association with the optional active agent). A particle of the present invention comprising a protein and an active agent may have improved (e.g., increased) storage, stability, activity, and/or function compared to the storage, stability, activity, and/or function of the protein alone (e.g., the protein not present in a particle of the present invention and not in association with the active agent). In some embodiments, upon storage at about 4° C. to about 10° C. in a closed container for about 1, 2, 3, 4, 5, or 6 month(s), the size (e.g., diameter) in at least one dimension of the particle remains within ±about 20% of its original size (e.g., the size at initial formation of the particle and/or the size at day 1 of storage). For example, at an initial time point (e.g., the start of day one of the storage time period), the particle may have a diameter of about 125 nm and after storage at about 4° C. to about 10° C. in a closed container for about 1, 2, 3, 4, 5, or 6 month(s) starting from day one of the storage time period, the particle may have a size that increased or decreased by about 20% or less. Thus, the particle having a starting size of about 125 nm may have a size at the end of the storage time period in a range of about 100 nm to about 150 nm. In some embodiments, upon storage at about 4° C. to about 10° C. in a closed container for about 1, 2, 3, 4, 5, or 6 month(s), a particle of the present invention has a size (e.g., diameter) in at least one dimension that is increased in an amount of less than about 20% compared to its original size. In some embodiments, following heating (e.g., heating at a temperature of about 50° C., 55° C., 60° C., 65° C., 70° C., 75° C., 80° C., or 85° C., for a period of time of about 10 minutes, 20 minutes, or 30 minutes to about 60 minutes, 90 minutes, or 120 minutes), the size (e.g., diameter) in at least one dimension of the particle remains within ±about 20% of its original size (e.g., the size at initial formation of the particle and/or the size immediately prior to heating). In some embodiments, at an initial time point prior to heating, the particle may have a diameter of about 125 nm and after heating at a temperature of about 50° C., 55° C., 60° C., 65° C., 70° C., 75° C., 80° C., 85° C., or 90° C., for a period of time of about 2 minutes, 5 minutes, 10 minutes, 20 minutes, or 30 minutes to about 60 minutes, 90 minutes, or 120 minutes, the particle may have a size that increased or decreased by about 20% or less. In some embodiments, a dried particle (e.g., a freeze-dried and/or spray-dried particle and/or a particle that comprises water in an amount of about 0% to about 5% by weight of the dried particle) is stored at about 4° C. to about 10° C. in a closed container for about 1, 2, 3, 4, 5, or 6 month(s) and optionally, at the end of the storage period, the size (e.g., diameter) of the dried particle is measured and/or the dried particle is re-constituted (e.g., dissolved and/or dispersed in) in a composition (e.g., water and/or a buffer) and the size (e.g., diameter) of the particle in the composition is measured. In some embodiments, a particle present in a composition (e.g., water and/or a buffer) is stored at about 4° C. to about 10° C. in a closed container for about 1, 2, 3, 4, 5, or 6 month(s) and optionally the size (e.g., diameter) of the particle in the composition is measured at the end of the storage time period.

In some embodiments, a particle of the present invention and/or a plurality of particles of the present invention is stable in that it has one peak particle size distribution in a composition at a pH of about 3, a composition at a pH of about 7, and/or in a composition at a pH of about 11, optionally as measured using microscopy (e.g., SEM and/or TEM) and/or DLS. In some embodiments, a particle of the present invention and/or a plurality of particles of the present invention is stable in that it has one peak particle size distribution in a composition at a pH of about 3, one peak particle size distribution in a composition at a pH of about 7, and one peak particle size distribution in a composition at a pH of about 11, optionally as measured using microscopy (e.g., SEM and/or TEM) and/or DLS. In some embodiments, a composition (e.g., a composition having a pH of about 3, 7, and/or about 11) comprising a plurality of particles has two or more peak particle size distributions, optionally as measured using microscopy (e.g., SEM and/or TEM) and/or DLS, which indicates that the particles of the plurality of particles are not stable. In some embodiments, a particle of the present invention is stable in that the particle does not fall out of (e.g., precipitate) and/or aggregate in a composition of the present invention. In some embodiments, a particle of the present invention is stable in that the particle does not fall out of (e.g., precipitate) and/or aggregate in a composition of the present invention when the pH of the composition is adjusted (e.g., from a pH of about 11 to a pH of about 3).

In some embodiments, a particle of the present invention and/or a protein therein has an increased activity and/or function (e.g., increased antioxidant activity) compared to the activity and/or function of the protein that has not been provided in a particle of the present invention. In some embodiments, a particle of the present invention has an improved function. In some embodiments, a particle of the present invention improves a property of the protein and/or the active agent present in the particle. For example, in some embodiments, a particle of the present invention may reduce the bitterness of an active agent (e.g., tryptophan) such as by increasing the bitter taste threshold (BTT), optionally as measured by Di Pizio & Nicoli, 2020 Molecules (Basel, Switzerland), 25 (20), doi.org/10.3390/molecules25204623, which is incorporated herein for methods of measuring bitterness and/or BTT. For example, a particle of the present invention may provide a BTT of about 4 mmol/L, 5 mmol/L, or 6 mmol/L to about 7 mmol/L or 8 mmol/L for an active agent.

According to some embodiments, a composition comprising a particle of the present invention is provided. In some embodiments, the composition comprises a plurality of particles of the present invention. In some embodiments, the composition comprises a particle of the present invention and a carrier. The carrier may be a liquid such as, but not limited to, water and/or an oil. In some embodiments, the carrier is a food-grade component such as, but not limited to, an alcohol (e.g., ethanol such as in an amount of about 5% to about 20%). One or more excipient(s) may be present in a composition of the present invention. Exemplary excipients include, but are not limited to, pectins and/or gums. In some embodiments, a composition of the present invention is devoid of a masking agent, flavoring agent, cyclodextrin (e.g., β-cyclodextrin), and/or a physical barrier that is optionally configured to mask or reduce the taste of an active agent that may be present in a particle of the present invention. In some embodiments, the composition is not a gel (e.g., a hydrogel such as a protein hydrogel) and/or is not an emulsion. In some embodiments, a particle of the present invention is not present in a gel (e.g., a hydrogel such as a protein hydrogel) or an emulsion. In some embodiments, a composition of the present invention is devoid of an agent (e.g., masking agent) that is configured and/or designed to reduce bitterness and/or off-taste of an active agent that may be present in the composition rather than configured and/or designed for providing the desired flavor or taste of the composition.

In some embodiments, a composition of the present invention is a food product, nutritional supplement, therapeutic drink, and/or cosmetic. In some embodiments, a particle of the present invention may be present in a food product. In some embodiments, the food product is a dairy product (e.g., milk, yogurt, etc.). Particles may be present in a composition and/or article of the present invention in an amount of about 1%, 2%, 3%, 4%, 5%, 10%, 15%, 20%, 25%, or 30% by weight of the composition and/or article.

An active agent, when present in a particle of the present invention, may remain associated with (e.g., complexed with, within, etc.) the particle and/or protein present in the particle when present in a carrier and/or composition of the present invention. In some embodiments, about 30% or less of the total amount of active agent added to a carrier and/or composition is present free (i.e., not associated with the particle and/or protein) in the carrier and/or composition. For example, about 0%, 1%, 2%, 5%, 10%, or 15% to about 20%, 25%, or 30% of the active agent present in a particle of the present invention that is provided in a carrier and/or composition and/or that is used to prepare a particle of the present invention may be present in the carrier and/or composition as free active agent (i.e., active agent that is not associated with the particle and/or protein). Thus, for a particle comprising the active agent in a given amount, when the particle is added to a carrier and/or composition, the amount of free active agent present in the carrier and/or composition may be about 30% or less than the given amount of the active agent present in the particle. In some embodiments, a composition comprising water and particles of the present invention, which particles are present in an amount of about 100 mg of particles per mL of water, comprises free active agent in an amount of about 0%, 1%, 2%, 5%, 10%, or 15% to about 20%, 25%, or 30% by weight of the total amount of the active agent present in the particles. A composition of the present invention may be a dispersion (e.g., a colloidal dispersion). In some embodiments, a composition of the present invention has no visible aggregation in the composition (e.g., no visible clumps, aggregates, or particulates). In some embodiments, the composition is clear and is not cloudy or opaque. In some embodiments, the composition may appear turbid, but no sediments and/or aggregations are present.

Provided according to some embodiments of the present invention is a method for preparing a particle of the present invention. In some embodiments, a method of the present invention comprises providing a protein in a composition (e.g., a protein dissolved and/or suspended in an aqueous composition (e.g., solution)); adjusting the pH of the composition to a basic pH, then adjusting the pH of the composition to an acidic pH; and optionally, then heating the composition, thereby forming the particle that comprises the protein. In some embodiments, the particle is devoid of an active agent. In some embodiments, the particle comprises the protein and an active agent. In some embodiments, a method of the present invention comprises combining a protein and an active agent to form a mixture; adjusting the pH of the mixture to a basic pH, then adjusting the pH of the mixture to an acidic pH; and optionally, then heating the mixture, thereby forming a particle that comprises the protein and the active agent.

In some embodiments, a composition comprising a protein is an aqueous composition. In some embodiments, a mixture comprising a protein and an active agent is an aqueous composition. In some embodiments, a protein and optionally an active agent may be dissolved in the aqueous composition that is used in a method of the present invention. In some embodiments, the protein and/or the active agent may be dissolved in the aqueous composition. In some embodiments, a mixture used in a method of the present invention comprises a protein and an active agent in a weight ratio of about 2:1, 3:1, 4:1, 5:1, 6:1, 7:1, 8:1, 9:1, 10:1, 11:1, 12:1, 13:1, 14:1, or 15:1 to about 16:1, 17:1, 18:1, 19:1, 20:1, 21:1, 22:1, 23:1, 24:1, 25:1, 26:1, 27:1, 28:1, 29:1, or 30:1 (protein:active agent). In some embodiments, the mixture comprises the protein and the active agent in a weight ratio of about 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, or 30:1 (protein:active agent). In some embodiments, the mixture comprises the protein and the active agent in a weight ratio of about 5:1 to about 20:1 (protein:active agent). In some embodiments, the mixture comprises the protein and the active agent in a weight ratio of about 5:1 (protein:active agent). In some embodiments, a mixture used in a method of the present invention has a total solids content in a range of about 1%, 2%, 3%, 4%, or 5% to about 6%, 7%, 8%, 9%, or 10% w/v. In some embodiments, the mixture has a total solids content of about 1%, 2%, 3%, 4%, 5%, 6%, 7%, 8%, 9%, or 10%. In some embodiments, the mixture has a total solids content of about 2.5% w/v.

In some embodiments, an active agent may be present in a composition (e.g., a mixture) used in a method of the present invention in an amount of about 0, 1, 5, 10, 15, 20, 25, 30, 35, 40, 45, or 50 mg/mL to about 55, 60, 65, 70, 75, 80, 85, 90, 95, or 100 mg/mL and/or a protein may be present in the composition an amount of about 1, 5, 10, 15, 20, 25, 30, 35, 40, 45, 50, 55, 60, 65, 70, 75, 80, 85, 90, 95, or 100 mg/mL to about 110, 120, 130, 140, 150, 160, 170, 180, 190, or 200 mg/mL. For example, in some embodiments, the active agent (e.g., tryptophan) may be dissolved in a mixture of the present invention in an amount of about 30 mg/mL to about 50 mg/mL and the protein (e.g., WPI) may be dissolved in the same mixture in an amount of about 50 or 75 mg/mL to about 100, 150, or 200 mg/L. In some embodiments, WPI is dissolved in a mixture of the present invention in an amount of about 200 mg/mL or less such as in an amount of about 50 mg/mL to about 100 mg/mL. In some embodiments, WPI is dissolved in a mixture of the present invention in an amount of about 50 mg/mL or less, such as in an amount of about 10 mg/mL to about 50 mg/mL.

In some embodiments, adjusting the pH of a composition (e.g., a mixture) used in a method of the present invention to a basic pH comprises adjusting the pH of the composition to a pH of about 8, 9, or 10 to about 11, 12, or 13. In some embodiments, adjusting the pH of the composition to a basic pH comprises adjusting the pH of the composition to a pH of about 11. In some embodiments, adjusting the pH of a composition used in a method of the present invention to an acidic pH comprises adjusting the pH of the composition to a pH of less than about 7, 6, 5, 4, 3, or 2. In some embodiments, adjusting the pH of the composition to an acidic pH comprises adjusting the pH of the composition to a pH of about 2, 2.5, 3, 3.5, 4, 4.5, or 5 to about 5.5, 6, or 6.5. In some embodiments, adjusting the pH of the composition to an acidic pH comprises adjusting the pH of the composition to a pH of about 2, 2.5, 3, 3.5, 4, 4.5, 5, 5.5, 6, or 6.5. In some embodiments, adjusting the pH of the composition to an acidic pH comprises adjusting the pH of the composition to a pH of about 6. In some embodiments, a method of the present invention comprises adjusting the pH of a composition of the present invention to a pH of about 11, then adjusting the pH of the composition to a pH of about 6.

An acid and/or base (e.g., an organic or inorganic acid and/or an organic or inorganic base, respectively) may be used to adjust the pH of a composition of the present invention. In some embodiments, adjusting the composition to a basic pH may comprise adding a base to the composition. In some embodiments, the base is sodium hydroxide (NaOH) and/or a NaOH solution. In some embodiments, adjusting the pH of the composition to an acidic pH comprises adding an acid to the composition. In some embodiments, the acid is hydrochloric acid (HCl) and/or a HCl solution.

In some embodiments, adjusting the composition to an acidic pH comprises adding an acid to the composition via dropwise addition, optionally while mixing the composition. In some embodiments, optionally while mixing the composition, adjusting the composition to an acidic pH comprises adding an acid to the composition via a method in which less than the total volume of the acid is added to the composition in a single addition of the acid such that the total volume of the acid is added over a period of time in two or more additions rather than adding the total volume of the acid in a single addition. In some embodiments, two or more (e.g., 2, 4, 6, 8, 10, or more) additions of an acid that are each less than the total volume of the acid that is added to the composition are added to a composition during the adjusting and/or method, optionally while mixing the composition. In some embodiments, adjusting the composition to an acidic pH comprises adding an acid to the composition via a single addition of the total volume of the acid into the composition, optionally while mixing the composition. In some embodiments, adjusting the composition to an acidic pH may comprise adding hydrochloric acid (HCl) and/or a HCl solution to the composition via dropwise addition, optionally while mixing the composition. In some embodiments, optionally while mixing the composition, adjusting the composition to an acidic pH comprises adding HCl and/or a HCl solution to a composition via two or more additions of HCl and/or HCl solution that are each less than the total volume of the HCl and/or HCl solution that is added to the composition during the adjusting and/or method. In some embodiments, adjusting the composition to an acidic pH may comprise adding HCl and/or a HCl solution to the composition via direct mixing of the total volume of the HCl and/or a HCl solution into the composition. In some embodiments, a method of the present invention does not comprise (i.e., is devoid of) heating a composition (e.g., a mixture) of the present invention and comprises adjusting the composition to an acidic pH comprising adding an acid to the composition via dropwise addition and/or via two or more (e.g., 2, 4, 6, 8, 10, or more) additions of an acid that are each less than the total volume of the acid that is added to the composition during the adjusting and/or method. In some embodiments, a method of the present invention does not comprise heating a composition of the present invention and comprises adjusting the composition to an acidic pH comprising adding an acid to the composition via direct mixing of the total volume of the acid into the composition.

In some embodiments, adjusting the pH of the composition to an acidic pH occurs about 15, 20, or 25 minutes to about 30, 35, or 40 minutes after the adjusting of the pH of the composition to a basic pH. In some embodiments, adjusting the pH of the composition to an acidic pH occurs about 30 minutes after the adjusting of the pH of the composition to a basic pH. In some embodiments, responsive to adjusting the pH of the composition to a basic pH, the composition may be mixed at the basic pH for about 15, 20, or 25 minutes to about 30, 35, or 40 minutes. In some embodiments, responsive to adjusting the pH of the composition to a basic pH, the composition may be mixed at the basic pH for about 30 minutes. In some embodiments, responsive to adjusting the pH of the composition to an acidic pH, the composition may be mixed at the acidic pH for about 15, 20, or 25 minutes to about 30, 35, or 40 minutes. In some embodiments, responsive to adjusting the pH of the composition to an acidic pH, the composition may be mixed at the acidic pH for about 30 minutes.

In some embodiments, a method of the present invention comprises heating a composition (e.g., a mixture) of the present invention. In some embodiments, heating a composition (e.g., a mixture) in a method of the present invention comprises heating the composition for about 10, 15, 20, 25, or 30 minutes to about 35, 40, 45, 50, 55, or 60 minutes. In some embodiments, a method of the present invention comprises heating the composition for about 10, 15, 20, 25, 30, 35, 40, 45, 50, 55, or 60 minutes. In some embodiments, a method of the present invention comprises heating the composition for about 20 minutes. In some embodiments, a method of the present invention comprises heating the composition to a temperature in a range of about 50, 55, or 60° C. to about 65, 70, 75, or 80° C. In some embodiments, a method of the present invention comprises heating the composition to a temperature of about 50, 55, 60, 65, 70, 75, or 80° C. In some embodiments, a method of the present invention comprises heating the composition to a temperature of about 70° C. In some embodiments, a method of the present invention comprises heating the composition to a temperature in a range of about 50, 55, or 60° C. to about 65, 70, 75, or 80° C. and maintaining the temperature for about 10, 15, 20, 25, or 30 minutes to about 35, 40, 45, 50, 55, or 60 minutes. In some embodiments, a method of the present invention comprises heating the composition to a temperature of about 70° C. and maintaining the temperature for about 20 minutes. In some embodiments, a method of the present invention does not comprise (i.e., is devoid of) heating a composition of the present invention. In some embodiments, a method of the present invention does not comprise (i.e., is devoid of) heating a composition of the present invention to a temperature of about 50, 55, 60, 65, 70, 75, or 80° C. and/or maintaining the temperature for about 10, 15, 20, 25, or 30 minutes to about 35, 40, 45, 50, 55, or 60 minutes.

In some embodiments, a method of the present invention comprises combining a protein (e.g., a whey protein isolate) and an active agent (e.g., tryptophan) to form a mixture; adjusting the pH of the mixture to a pH of about 11, mixing (e.g., shaking, stirring) the mixture for about 10, 20, 30, 40, 50, or 60 minutes, and then adjusting the pH of the mixture to a pH of about 6; and heating the mixture to a temperature of about 50° C., 60° C., 70° C., or 80° C. for about 1, 2, 3, 4, 5, 10, 20, 30, 40, 50, or 60 minutes. In some embodiments, the mixture comprises the protein and the active agent in a ratio of about 5:1 w/w (protein:active agent, e.g., whey protein isolate:tryptophan). In some embodiments, the mixture has a total solids content of about 2.5% w/v. In some embodiments, the protein is whey protein isolate. In some embodiments, the active agent is tryptophan. In some embodiments, adjusting the pH of the mixture to a pH of about 11 comprises adding sodium hydroxide (NaOH) and/or an NaOH solution to the mixture. In some embodiments, mixing (e.g., shaking, stirring) the mixture is for about 20 minutes. In some embodiments, adjusting the pH of the mixture to a pH of about 6 comprises adding hydrochloric acid (HCl) and/or an HCl solution to the mixture. In some embodiments, heating the mixture is at a temperature of about 70° C. for about 1 minute.

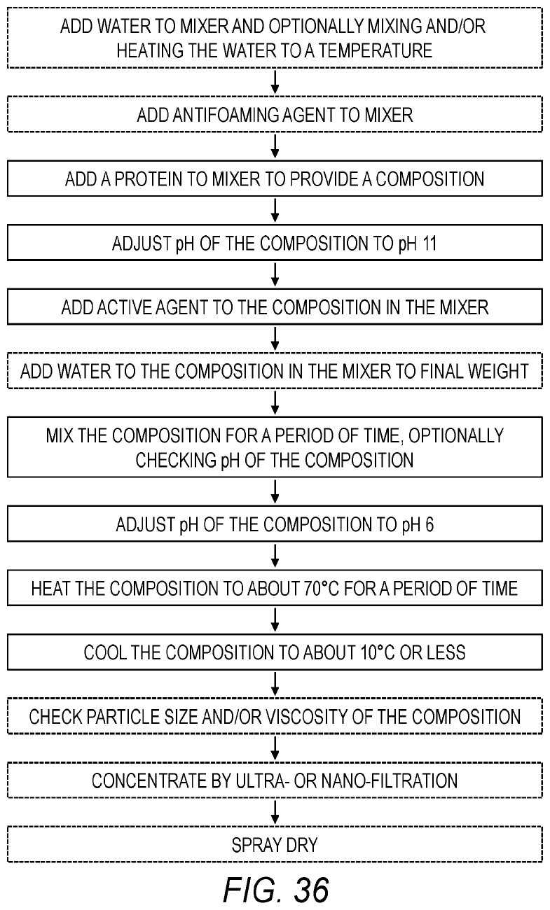

As shown in FIG. 36, in some embodiments a method of the present invention comprises optionally adding water to a mixer and optionally mixing and/or heating the water to a temperature; optionally adding an antifoaming agent (e.g., a food grade antifoam agent such as MAGRABAR® PD-602, a silicone antifoam agent, and/or a vegetable oil based antifoam agent) to the mixer; adding a protein (e.g., a whey protein isolate) to the mixer to provide a composition; adjusting the pH of the composition to a pH of about 11; adding an active agent (e.g., tryptophan) optionally dissolved and/or suspended in an aqueous composition (e.g., solution)) to the composition in the mixer; optionally adding water to the composition to a final weight; mixing the composition for a period of time, optionally checking the pH of the composition and, if needed, adjusting (e.g., adjusting to a pH of about 11); adjusting the pH of the composition to a pH of about 6; heating the composition to a temperature of about 70° C. and maintaining the temperature for a period of time, then cooling the composition to a temperature of about 10° C. or less; optionally checking the particle size of particles in the composition and/or viscosity of the composition; optionally concentrating the composition using ultra-filtration or nano-filtration; and optionally spray drying the composition. In some embodiments, the mixer is an in-line mixer. In some embodiments, the method comprises adding water to the mixer and mixing while heating the water to a temperature of about 20, 21, 22, 23, 24, or 25° C., optionally to a temperature of about 21° C. In some embodiments, the method comprises adding an antifoaming agent to the mixer in an amount of about 30, 31, 32, 33, 34, 35, 36, 37, 38, 39, or 40 ppm antifoaming agent, optionally about 35 ppm antifoaming agent. In some embodiments, the method comprises adding a protein (e.g., whey protein isolate) to the mixer in an amount of about 1, 2, 3, 4, or 5% w/v, optionally in an amount of about 4% w/v. In some embodiments, the protein is whey protein isolate. In some embodiments, the method comprises adjusting the pH of the composition to a pH of about 11 by adding sodium hydrochloride (NaOH) and/or an NaOH solution to the composition, optionally wherein the NaOH is 1N NaOH. In some embodiments, the method comprises adding an active agent (e.g., tryptophan) in an amount of about 0.5, 0.6, 0.7, 0.8, 0.9, or 1.0% w/v, optionally in an amount of about 0.8% w/v. In some embodiments, the active agent is tryptophan. In some embodiments, mixing the composition for a period of time comprises mixing the composition for about 10, 15, 20, 25, or 30 minutes. In some embodiments, adjusting the pH to a pH of about 6 comprises adding hydrochloric acid (HCl) and/or an HCl solution to the composition, optionally wherein the HCl is 1N HCl, and optionally mixing the composition for about 1, 2, 3, 4, or 5 minutes, optionally mixing the composition having at a pH of about 6 for about 2 minutes. In some embodiments, the method comprises heating the composition to a temperature of about 70° C. for about 10, 20, 30, 40, or 50 seconds to about 1, 2, 3, 4, or 5 minutes, optionally for about 1 minute. In some embodiments, the method comprises checking the particle size of particles in the composition and/or checking the viscosity of the composition. In some embodiments, checking the particle size comprises checking that the mean particle size (e.g., mean particle diameter) of the particles in the composition is about 100, 125, 150, 175, 200, 250, 300, 350, 400, or 450 nm, optionally about 400 nm. In some embodiments, the method comprises concentrating the composition using ultra-filtration or nano-filtration. In some embodiments, the composition is concentrated by an ultra-filtration method, optionally using a four inch 10 kDa membrane. In some embodiments, the composition is concentrated using a nano-filtration method, optionally using a four inch 300 Dalton membrane. In some embodiments, a method of the present invention comprises removing (e.g., filtering out) particles that are smaller than 10 kDa. In some embodiments, a method of the present invention comprises removing salts and/or free amino acids (e.g., free tryptophan) from a composition of the present invention. A method of the present invention may produce a particle of the present invention having a particle size distribution from about 50, 100, 200, or 300 nm to about 400, 500, 600, or 700 nm. In some embodiments, the particle may have a polydispersity index of less than about 0.5, 0.45, 0.4, 0.35, 0.3, 0.25, or 0.2. In some embodiments, the particle may have a polydispersity index of about 0.2. In some embodiments, the particle may have a mean particle size (e.g., mean particle diameter) of about 50, 75, or 100 nm to about 125, 150, or 200 nm. In some embodiments, the particle may have a mean particle size (e.g., mean particle diameter) of about 50, 75, 100, 125, 150, or 200 nm. In some embodiments, the particle may have a mean particle size (e.g., mean particle diameter) of about 125 nm. In some embodiments, the particle may have a mean particle size (e.g., mean particle diameter) of about 110 nm. In some embodiments, the particle has a free sulfhydryl (SH) group content of about 5, 6, 7, 8, 9, or 10 μmol SH/g to about 11, 12, 13, 14, or 15 μmol SH/g, optionally as measured using Ellman's reagent (5,5′-dithiobis-(2-nitrobenzoic acid) or DTNB) and UV-Vis spectroscopy. In some embodiments, a method of the present invention may produce a particle of the present invention having an increased sulfhydryl (SH) group content compared to the sulfhydryl (SH) group content of a particle not produced according to a method of the present invention, optionally as measured using Ellman's reagent (5,5′-dithiobis-(2-nitrobenzoic acid) or DTNB) and UV-Vis spectroscopy. In some embodiments, the particle may have a surface hydrophobicity of about 4×10⁷ to about 7×10⁷, optionally as measured using 8-anilino-1-naphthalene sulfonate (ANS) as a fluorescence probe. In some embodiments, a method of the present invention may produce a particle of the present invention having an increased surface hydrophobicity compared to the surface hydrophobicity of a particle not produced according to a method of the present invention, optionally as measured using 8-anilino-1-naphthalene sulfonate (ANS) as a fluorescence probe. In some embodiments, a method of the present invention may produce a particle of the present invention having an increased fluorescence intensity compared to the fluorescence intensity of a particle not produced according to a method of the present invention, optionally as measured by fluorescence spectroscopy.

In some embodiments, the active agent is present in the particle in an amount of about 0%, 5%, or 10% to about 15%, 20%, or 25% by weight of the particle. In some embodiments, the active agent is present in the particle in an amount of about 0%, 5%, 10%, 15%, 20%, or 25% by weight of the particle. In some embodiments, the protein is present in the particle in an amount of about 75%, 80%, or 85% to about 90%, 95%, or 100% by weight of the particle. In some embodiments, the protein is present in the particle in an amount of about 75%, 80%, 85%, 90%, 95%, or 100% by weight of the particle. In some embodiments, the active agent is present in the particle in an amount of about 0%, 5%, or 10% to about 15%, 20%, or 25% by weight of the particle and the protein is present in the particle in an amount of about 75%, 80%, or 85% to about 90%, 95%, or 100% by weight of the particle.

In some embodiments, a method of the present invention comprises dehydrating a particle of the present invention and/or a composition in which the particle is present. Dehydrating a particle of the present invention and/or a composition comprising a particle of the present invention may be carried out using methods and/or devices known in the art. In some embodiments, dehydrating a particle of the present invention and/or a composition comprising a particle of the present invention comprises freeze-drying and/or spray-drying the particle and/or composition. In some embodiments, dehydrating a particle of the present invention comprises freeze-drying and/or spray-drying the composition following optional heating.

A method of the present invention may comprise reducing the size (e.g., diameter) of a particle of the present invention. For example, in some embodiments, the size (e.g., diameter) of a particle of the present invention may be reduced upon adjusting the pH of a composition in which the particle is present. In some embodiments, a method of the present invention comprises reducing the size (e.g., diameter, optionally the average diameter) of a particle of the present invention by about 5%, 10%, 15%, or 20% to about 25%, 30%, 35%, or 40% compared to the size of the particle in a composition at a pH of about 11. In some embodiments, the size (e.g., diameter, optionally the average diameter) of a particle of the present invention present in a composition may be reduced by about 5%, 10%, 15%, or 20% to about 25%, 30%, 35%, or 40% after the pH of the composition is adjusted from a pH of about 11 to a pH of about 6 or 3.

In some embodiments, a method of the present invention comprises administering a therapeutically effective amount of a particle of the present invention and/or a composition of the present invention to a subject. As used herein, the term “therapeutically effective amount” refers to an amount of particle and/or composition of the present invention that elicits a therapeutically useful response in a subject. Those skilled in the art will appreciate that the therapeutic effects need not be complete or curative, as long as some benefit is provided to the subject.

“Treat,” “treating” or “treatment of” (and grammatical variations thereof) as used herein refer to any type of treatment that imparts a benefit to a subject and may mean that the severity of the subject's condition is reduced, at least partially improved or ameliorated and/or that some alleviation, mitigation or decrease in at least one clinical symptom associated with the subject's condition is achieved and/or there is a delay in the progression of the symptom. In some embodiments, the severity of a symptom associated with sleep quality and/or mental health may be reduced in a subject compared to the severity of the symptom in the absence of a method of the present invention. In some embodiments, a particle of the present invention and/or a composition of the present invention is administered to a subject to improve sleep quality (e.g., increase the length of sleep time and/or time in rapid eye movement (REM) sleep, reduces sleep interruptions, etc.), improve mental health, and/or treat a disease and/or a symptom thereof.

In some embodiments, a particle of the present invention and/or a composition of the present invention may be administered in a treatment effective amount. A “treatment effective” amount as used herein is an amount that is sufficient to treat (as defined herein) a subject. Those skilled in the art will appreciate that the therapeutic effects need not be complete or curative, as long as some benefit is provided to the subject. In some embodiments, a treatment effective amount may be achieved by administering a composition of the present invention.

The terms “prevent,” “preventing” and “prevention” (and grammatical variations thereof) refer to avoidance, reduction and/or delay of the onset of a symptom associated with a disease, disorder, or condition and/or a reduction in the severity of the onset of symptom associated with a disease, disorder, or condition relative to what would occur in the absence of a method of the present invention. The prevention can be complete, e.g., the total absence of the symptom. The prevention can also be partial, such that the occurrence of the symptom in the subject and/or the severity of onset is less than what would occur in the absence of a method of the present invention. In some embodiments, a particle of the present invention and/or a composition of the present invention is administered to a subject to prevent a disease, disorder, or condition.

In some embodiments, a particle of the present invention and/or a composition of the present invention may be administered in a prevention effective amount. A “prevention effective” amount as used herein is an amount that is sufficient to prevent (as defined herein) a symptom associated with a disease, disorder, or condition in a subject. Those skilled in the art will appreciate that the level of prevention need not be complete, as long as some benefit is provided to the subject. In some embodiments, a prevention effective amount may be achieved by administering a composition of the present invention.

The present invention finds use in both veterinary and medical applications. Subjects suitable to be treated with a method of the present invention include, but are not limited to, mammalian subjects. Mammals of the present invention include, but are not limited to, canines, felines, bovines, caprines, equines, ovines, porcines, rodents (e.g. rats and mice), lagomorphs, primates (e.g., simians and humans), non-human primates (e.g., monkeys, baboons, chimpanzees, gorillas), and the like, and mammals in utero. Any mammalian subject in need of being treated according to the present invention is suitable. Human subjects of both genders and at any stage of development (i.e., neonate, infant, juvenile, adolescent, adult) may be treated according to the present invention. In some embodiments of the present invention, the subject is a mammal and in certain embodiments the subject is a human. Human subjects include both males and females of all ages including fetal, neonatal, infant, juvenile, adolescent, adult, and geriatric subjects as well as pregnant subjects. In particular embodiments of the present invention, the subject is a human adolescent and/or adult.

A method of the present invention may also be carried out on animal subjects, particularly mammalian subjects such as mice, rats, dogs, cats, livestock and horses for veterinary purposes, and/or for drug screening and drug development purposes.

In some embodiments, the subject is “in need of” or “in need thereof” a method of the present invention, for example, the subject has findings typically associated with a disease, disorder, or condition, is suspected to have a disease, disorder, or condition, and/or the subject has a disease, disorder, or condition.

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

### Example 1

### 1. Introduction

Tryptophan (Trp) was encapsulated in whey protein nanoparticles using a combination of pH shifting and thermal treatment. The combined effect of pH shifting and thermal treatment on the fabrication of whey protein isolate-tryptophan nanoparticles (WPI-Trp-NPs) was investigated. WPI-Trp particles had an average particle size of 110.1 nm and a low average PDI of 0.20. Fluorescence spectroscopy confirmed the encapsulation of Trp by WPI which shows higher fluorescence when the Trp is encapsulated by the WPI. Surface hydrophobicity, circular dichroism, particle size, free sulfhydryl, and antioxidant activity were used to characterize the WPI-Trp-NPs. The driving force for the complexation of WPI-Trp NPs was non-covalent bonding, such as hydrophobic interactions and hydrogen bonding. Molecular docking results indicated the formation of WPI-Trp nanocomplexes with alpha-lactalbumin (α-LA), bovine serum albumin and beta-lactoglobulin (β-LG) was hydrophobic in nature with hydrogen bonding affinity between Trp and these proteins where the strength of the interactions were ranked as α-LA>BSA>β-LG. The combination of pH-shifting and heating was an important method to improve the functionalities of WPI and an effective way to fabricate WPI-Trp nanoparticles.

### 2. Materials and Methods

### 2.1. Materials

Whey protein isolate powder (Provon 190) was provided by Glanbia Nationals, Inc (Fitchburg, WI, USA). Tryptophan (Trp, reagent grade >98%), 2,2-diphenyl-1-picrylhydrazyl (DPPH, reagent grade >97%), 2,2-azinobis(3-ethylbenzothiazoline-6-sulfonic acid (ABTS, reagent grade >98%), 8-Anilino-1-naphthalenesulphonate (ANS, reagent grade >98%), were purchased from Sigma-Aldrich (10417, St Louis, MO, USA). 5,5′-Dithiobis-(2-nitrobenzoic acid) (DTNB) was purchased from ThermoFisher (02451, Waltham, MA, USA) Hydrochloric acid (reagent grade >37%) and sodium hydroxide (reagent grade >97%) were purchased from Fisher Scientific (Hampton, NH, USA). Milli-Q water was prepared from a Millipore water purification system (Millipore Sigma, Burlington, MA, USA). All the other chemicals used in this study were analytical grade.

### 2.2 Preparation of Whey Protein-Tryptophan Nanoparticles (WPI-Trp NPs)

### 2.2.1 pH Shifting Treatment

The WPI-Trp solution was prepared by dissolving WPI and Trp powder samples in a 5:1 ratio (w/w) in Milli-Q water with magnetic stirring (800 rpm) for 2 h. The WPI-Trp solution was stored in a refrigerator (4° C.) for 12 h to ensure complete hydration. Next, the WPI-Trp solution was adjusted to pH 11 with 1 mol L⁻¹ NaOH and was stirred at 800 rpm for 1 h. Then, the WPI-Trp solution at pH 11 was divided into three parts and further readjusted into pH 5, 6, and 7 with 1 mol L⁻¹ HCl and stirred at 800 rpm for another hour. The resultant WPI-Trp solutions were adjusted to a final concentration of 2.5 w/v % with Milli-Q water to produce a pH shifting WPI-Trp stock solution. The WPI-Trp solution without pH shifting was divided into three parts and adjusted to pH 5, 6 and 7 with 1 mol L⁻¹ HCl, respectively, with a final concentration of 2.5 w/v %.

The control samples WPI and Trp with and without pH shifting were produced with the same protocol.

### 2.2.2 Heat Treatment on pH Shifting Samples

For the pH shifting combined with heating samples, aliquots of 2 mL pH-shifted WPI-Trp, WPI, and Trp sample solutions (2.5 w/v %) were filled into glass tubes separately and placed in a constant-temperature (50, 60, 70, 80° C.) water bath (Model WB20, VWR International) and heated for 10, 20, 30, 40, 50, and 60 min, respectively to induce heat-induced nanoparticles. The heat treatment was terminated by putting these glass tubes in an ice bath for 4 min to room temperature.

### 2.3 Characterization of WPI-Trp NPs

### 2.3.1 Particle Size Analysis

Particle size measurements were conducted to investigate the effect of pH shifting and heat treatment on protein aggregation. The WPI-Trp, WPI, and Trp nanoparticles were analyzed for their average diameter, particle size distribution, and polydispersity index (PDI) using dynamic light scattering instruments (Zetasizer Nano-ZS, Malvern, UK). The analyses were performed at 25° C. in a cuvette with 1 cm path. The measurements were repeated at least six times and in triplicate for each analysis.

### 2.3.2 Circular Dichroism (CD) Spectroscopy Analysis

CD spectroscopy was conducted using an AVIV-202-01 spectropolarimeter (Lakewood, NJ, USA) to investigate the secondary structure changes of WPI and WPI-Trp samples after thermal treatment and pH shifting. CD spectra were measured with a wavelength of 190-260 nm at 25° C. Samples with a concentration of 2 mg mL⁻¹ were measured in a 1-mm-path quartz cell. Web-based DichroWeb was used to calculate the secondary structures of samples.

### 2.3.3 Intrinsic Fluorescence

Intrinsic fluorescence spectra were carried out to study the aggregation-induced effect between WPI and Trp and the dynamic structure change of WPI. The intrinsic fluorescence of all the fabricated WPI-Trp, WPI, and Trp samples was recorded at room temperature using a fluorescence spectrophotometer (Hitachi, F-7000, Japan). Fluorescence emission spectra of the samples were excited at 295 nm and recorded from 310 to 500 nm (Zhan, F., et al. (2020). Food Hydrocolloids, 105, 105767).

### 2.3.4 Surface Hydrophobicity

Surface hydrophobicity measurements were conducted to investigate the protein conformational change during pH shifting and heating. Surface hydrophobicity was detected using ANS as a fluorescence probe (Jiang, H., et al., (2022). Food Chemistry, 393, 133358). The ANS stock solution (8.0 mM) was prepared in phosphate buffer (10 mM, pH 7.4). Six sample concentrations were prepared with the above buffer from 0.01 mg mL⁻¹ to 0.25 mg mL⁻¹. Next, 2 μL of ANS was added to 200 μL of WPI alone, and WPI-Trp mixture, with and without treatments (5:1, w/w) in a 96-well plate separately. Then, the plate was kept in the dark for 20 min before reading on a microplate reader (SpectraMax iD3, Molecular Devices, San Jose, CA, USA) with an excitation wavelength and an emission wavelength of 390 nm and 470 nm, respectively. The sample's surface hydrophobicity (H₀) was calculated as the slope of the curve of fluorescence intensity against sample concentration.

### 2.3.5 Turbidity

Turbidity was measured to analyze the development of protein aggregation during pH shifting and heating. The turbidity of WPI, Trp, and WPI-Trp was measured according to the previous method (Lin, T., et al. (2022). Food Hydrocolloids, 131, 107736).

### 2.3.6 Fourier Transform Infrared Spectroscopy (FTIR)

FTIR spectra were conducted to analyze the driving force for the formation of the WPI-Trp nanoparticles. The FTIR spectra of WPI, WPI-Trp, and Trp were recorded using the FTIR spectrometer (Shimadzu Instrument, Kyoto, Japan) in the range of 4000 cm⁻¹ to 400 cm⁻¹ wavenumbers. The measurement was taken in an average of 32 scans at a resolution of 4 cm⁻¹.

### 2.3.7 Scanning Electron Microscopy (SEM)

The microstructure of freeze-dried and solution samples was imaged using the SEM (Zeiss Gemini 500, Jena, Germany). Samples were vacuum-dried before scanning and imaging in the SEM. SEM analysis was done according to the previous method (Lin et al., 2022).

### 2.4 Antioxidant Activity

### 2.4.1 DPPH Radical Scavenging Capacity

The DPPH radical scavenging activity of the fabricated WPI, Trp and WPI-Trp samples was measured according to a previously published method (Dong, H., et al., (2016). International Journal of Biological Macromolecules, 93, 179-185) with a slight modification. The modifications are as follow: samples were diluted with Milli-Q water to various concentrations: 0.5 mg mL⁻¹, 1 mg mL⁻¹, 2 mg mL⁻¹ and 5 mg mL⁻¹. Next, 200 μl of sample was mixed with 100 μL of DPPH solution (0.2 mmol L⁻¹) and the mixture was stored in darkness for 30 min at room temperature (~25° C.).

### 2.4.2 ABTS Radical Scavenging Capacity

ABTS radical scavenging capacity was measured according to the previously published protocol with some modifications (Dong et al., 2016). The modifications are as follow: samples were dissolved in phosphate buffer (pH 7.4) to a concentration of 0.05 mg mL⁻¹. Next, a 200 μl aliquot of the sample was mixed with 20 μl of the ABTS working solution and the mixture was stored in dark for 20 min at room temperature (~25° C.).

### 2.5 Sulfhydryl Group Content (SH)

The free sulfhydryl group content of WPI and WPI-Trp samples were measured according to a previously published method of (Jiang, Z., et al., (2022). International Dairy Journal, 127, 105211). A mixture of samples with DTNB was considered as sample group, and samples without DTNB was considered as sample blank. Mixture of Tris-Gly buffer with DTNB was considered as reagent blank. The absorbance value of the mixture was recorded at 412 nm with a UV-Vis spectrophotometer (UV-2600, SHIMADZU Co., Japan). The content of free sulfhydryl groups was calculated according to the following equation:

$$SH ( µM {g}^{- 1} ) = \frac{( 73.53 \times D )}{C} \qquad ( 1 )$$

Where A₄₁₂ is the absorbance at 412 nm, C is the concentration of the sample, and D is the dilution factor.

### 2.6 Molecular Docking

Molecular docking was conducted to further understand the interaction mechanism of WPI and Trp. Auto Dock Vina 1.2.0 software (Trott & Olson, 2009) was performed to predict the interaction between whey protein and Trp. The 3D crystal structure of α-LA (PDB: 1F6R), β-LG (PDB: 3NPO), and BSA (PDB: 4F5S) were obtained from the web-based Protein Data Bank acting as receptor molecules that merge nonpolar hydrogens and incorporate charges. Blind docking was conducted to predict the lowest energy binding sites for Trp on α-LA, β-LG, and BSA. PyMOL ver. 2.5.2 software was used to visualize the docking results and the interactions between the ligand and receptor were analyzed by Ligplot (Wallace, A. C., Laskowski, R. A., & Thornton, J. M. (1995). LIGPLOT: A program to generate schematic diagrams of protein-ligand interactions. Protein Engineering, Design and Selection, 8 (2), 127-134.).

### 2.7 Data and Statistical Analysis

All data were analyzed and plotted by two software, JMP (version Pro15, SAS, USA) and GraphPad Prism9 (GraphPad Software Inc., USA). All the experiments were conducted in triplicate, and the mean value with standard deviation was expressed in the graphs. ANOVA and Tukey HSD comparison test (p<0.05) were used to analyze the sample differences.

### 3. Results and Discussion

### 3.1 Effect of pH Shifting on WPI-Trp Nanoparticle Formation

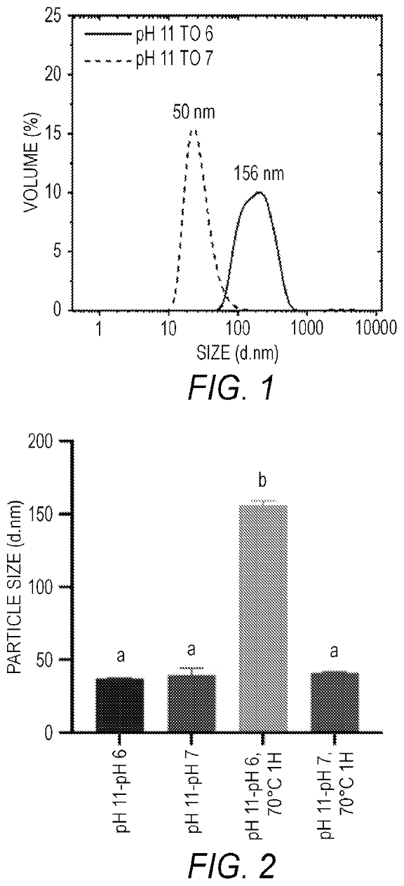

To determine the effect of pH shifting on the formation of WPI-Trp nanoparticles, 2.5 w/v % solutions of WPI and Trp were mixed at a ratio of 5:1 (w/w) and were dissolved in an alkaline solution at pH 11 before being shifted back to a pH of 5, 6, or 7, respectively, followed by heat treatment at 70° C. for 60 min. The sample with pH shifting from 11 to 5 showed undesirable precipitation (e.g., large aggregates and sediments), evidenced by high turbidity of the mixture, and therefore did not have adequate formation of WPI-Trp nanoparticles. Aggregation can occur at a pH of 5, even at room temperature, because pH 5 is close to the isoelectric point of WPI (pI 5.1), at which the protein carries a net zero charge. Aggregation is further enhanced by heat treatment. However, the samples with pH shifting from 11 to 7 or 11 to 6 showed colloidal dispersion without visible aggregates, indicating the formation of nanoparticles. This result was confirmed by the particle size distribution results as shown in FIG. 1. As shown in FIG. 1, as compared to the sample with pH shifting from 11 to 6, the sample with pH shifting from 11 to 7 showed a smaller particle size, indicating the formation of WPI-Trp nanoparticles. The particle sizes of WPI-Trp NPs obtained under pH shifting (from 11 to 7 and 11 to 6), and pH shifting combined heat treatment (pH shifting from 11 to 7 and 6 combined with heating under 70° C. for 1 h) ranged from less than 50.0 nm to the largest particles size of 156.0±3.0 nm when the pH was shifted from 11 to 6 followed by heating (FIG. 2). Without wishing to be bound to any particular theory, this may be attributed to the pH being near the iso-electric point of WPI and, thus, electrostatic repulsion was low. Shifting the pH from 11 to 7 followed by heating did not significantly increase (p>0.05) the particle size of WPI-Trp and the particles were smaller than the samples shifting from pH 11 to 6 followed by heating. This difference in size may be due to the fact that the heat-induced aggregation influence was much weaker at a pH further away from the isoelectric point.

Intrinsic fluorescence has been considered an effective approach to study the conformational changes of proteins. Tryptophan is one of the main fluorophores of protein, and the binding characteristics of Trp are studied by recording the fluorescence spectra emission at specific excitation wavelengths. Aggregation-induced emission is observed when a fluorescent molecule fluoresces when it is trapped in the protein aggregates as the rotation of the fluorescence molecule is restrained. The emission can be strongly quenched, or non-fluorescent, when it is free dispersed in buffer. Therefore, the intrinsic fluorescence intensity of Trp is positively related to the encapsulated Trp content in protein aggregates and can be used to characterize WPI-Trp nanoparticles. Without wishing to be bound to any particular theory, a schematic of a proposed mechanism of aggregation-induced fluorescence emission of Trp is shown in FIG. 4.

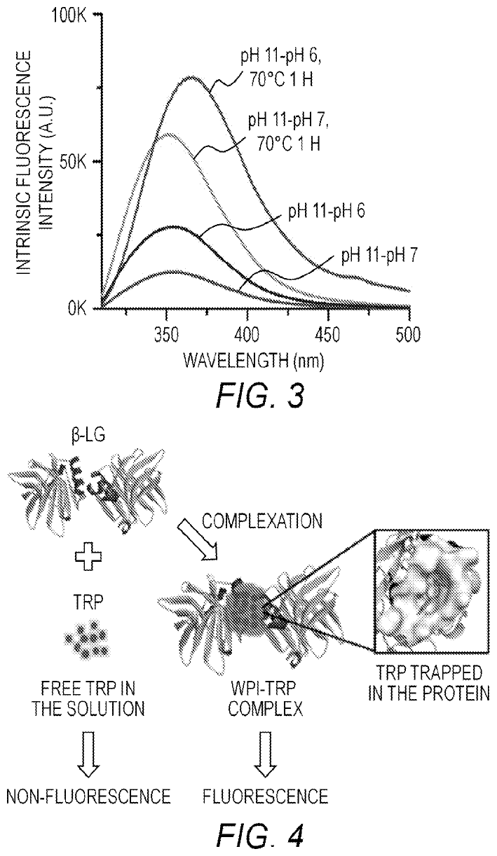

The fluorescence spectra of the WPI-Trp sample that was pH shifted from 11 to 6 showed a higher intrinsic fluorescence intensity than the sample that was pH shifted from 11 to 7 (FIG. 3). This suggested that a higher content of Trp was trapped in the samples obtained by shifting from pH 11 to pH 6. Without wishing to be bound to any particular theory, the aggregation-induced emission theory has shown that higher content of trapped fluorescent Trp is positively related to a higher intrinsic Trp fluorescence intensity.

The sample shifted from pH 11 to pH 6 and then heated at 70° C. not only showed a higher intrinsic intensity, but also the band was red-shifted compared to the heated or unheated pH 11 to pH 7 sample as well as the unheated pH 11 to pH 6 sample. This provides further evidence that pH shifting from 11 to 6 followed by heating may result in more protein unfolding and, therefore, more available binding sites for Trp to interact with and become encapsulated into nanoparticles. We found that the WPI-Trp mixture obtained with pH shifting and heating showed a greater extent of protein unfolding, and thus showed additional complexation of Trp.

### 3.2 Effect of Heating Temperature on WPI-Trp Nanoparticle Formation

Heating temperature is one of the primary factors that can influence protein particle size. Previous studies have shown that the denaturation temperature of WPI is between 65° C. and 70° C. (L. Zhang, et al., (2021) International Dairy Journal, 123, 105175). Heating temperature can affect the extent of protein denaturation. Denatured proteins with exposed hydrophobic residues and reactive sulfhydryl groups are prone to result in hydrophobic interactions and form disulfide bonds between protein particles.

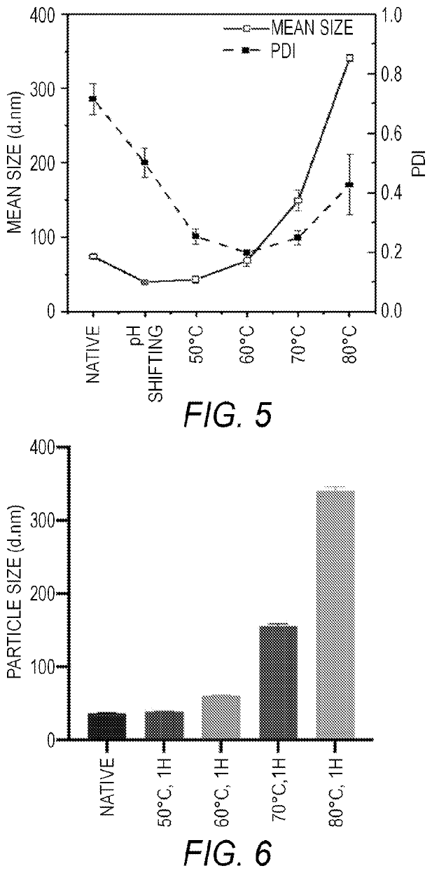

In our samples, higher temperature caused larger particles to form between WPI and Trp in a range from 38.9±0.6 nm at 50° C. to 341.1±5.1 nm at 80° C., as evidenced by a higher turbidity in the higher temperature samples, with the 80° C. sample having the highest turbidity. The effect of heating temperature on the particle size of WPI-Trp nanoparticles was performed by pH shifting from 11 to 6 followed by heat treatment at different temperatures for one hour. A sample of 2.5% (w/v) WPI-Trp at a 5:1 w/w ratio (WPI:Trp) was adjusted to a pH of 11, shaken for 30 minutes, adjusted to a pH of 6, and then underwent thermal treatment at either 50, 60, 70, or 80° C. for one hour. Clear differences were observed in the appearance of the WPI-Trp mixture after incubation at these different temperatures. WPI-Trp nanoparticles formed at 60° C. and 70° C. exhibited a colloidal suspension without any visible aggregation. When measuring the particle size of the colloidal suspension by DLS (FIG. 5), those samples produced by pH shifting and heat treatment at 60° C. and 70° C. showed a narrow particle size distribution (PDI<0.3) with a mean particle size of ~70 and ~150 nm, respectively. WPI-Trp pH shifted from pH 11 to 6 followed by heating at 80° C. for one hour produced the largest sized particles among the three temperature groups, which may be due to heat-induced aggregation. Increasing the temperature resulted in a red shift in the fluorescence spectra indicating that the proteins unfolded further upon increasing the temperature and more interior amino acid groups were exposed. Without wishing to be bound to any particular theory, it is believed that these exposed hydrophobic groups interact with additional Trp molecules which contribute to particle aggregation.

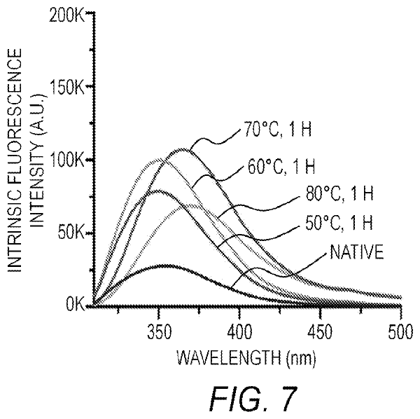

Additionally, as shown in FIG. 7, Trp fluorescence intensity increased with temperature from 50° C. to 70° C. and then decreased at 80° C., which may be due to a higher content of proteins that were unfolded followed by a strong self-assembly of proteins that limited Trp incorporation. By contrast, at 50° C. and 60° C. there may be less protein unfolding and aggregation and therefore a lack of exposed hydrophobic residues, limiting the encapsulation of Trp. This is consistent with the smaller particle size (FIG. 6).

### 3.3 Effect of Heating Time on WPI-Trp Nanoparticle Formation

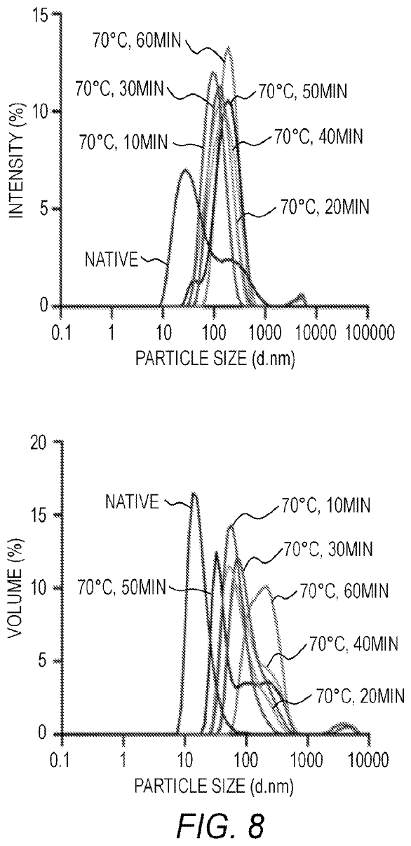

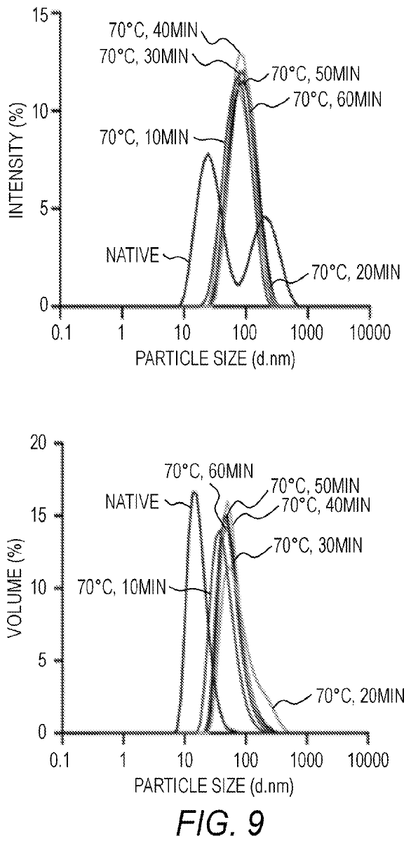

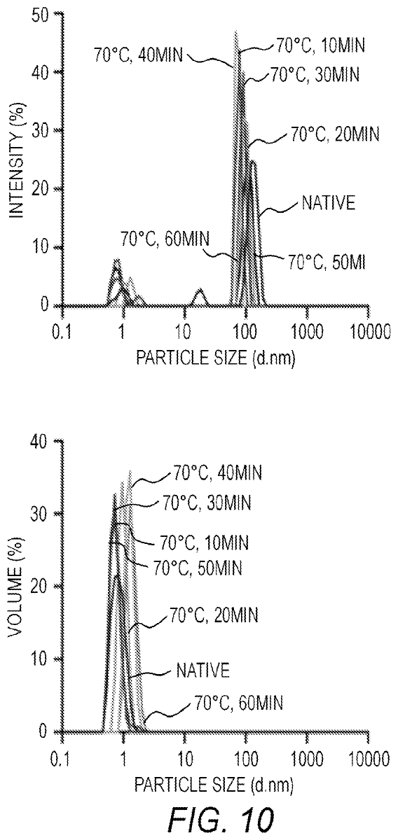

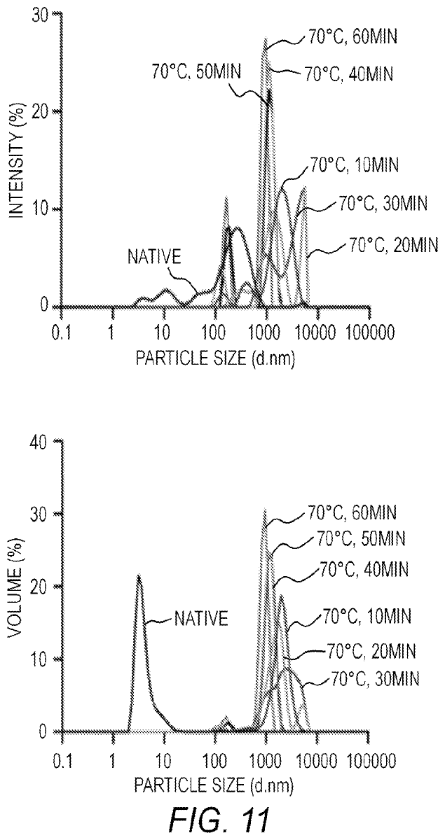

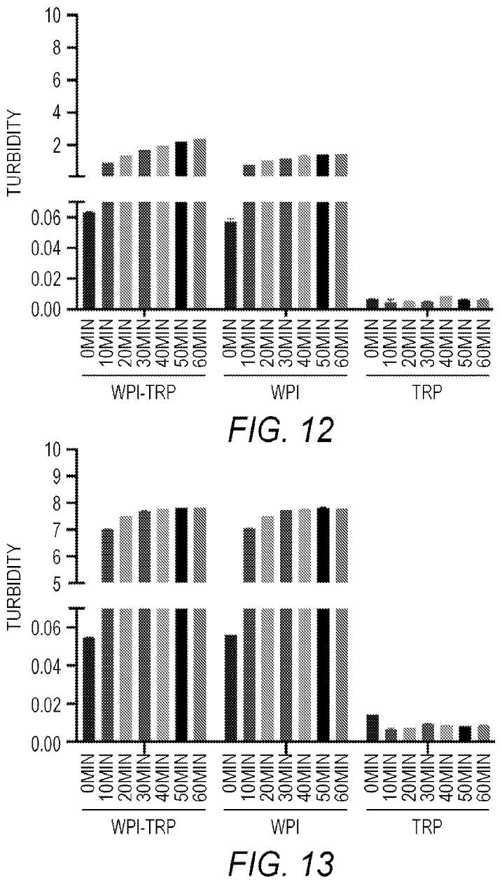

In addition to the temperature of heat treatments, heating time also influences protein aggregation. One of the primary indicators of the development of protein aggregation is turbidity. The effect of heating time on the particle size of WPI-Trp nanoparticles was investigated with and without pH shifting from 11 to 6 followed by heat treatment at 70° C. for different heating times between 0 and 60 minutes. Clear differences were observed in the appearance of the suspensions formed after heat treatment for each amount of time. With longer heating times, the WPI-Trp samples exhibited a more turbid suspension. Uniform nanoparticles were formed after heating for minutes (PDI<0.03). Both turbidity and particle size of the non-pH-shifted group and the pH-shifted group increased with longer heating times (FIGS. 8-13). WPI-Trp samples without pH shifting demonstrated significantly higher turbidity (FIG. 13) and larger particle sizes (FIG. 11) than samples obtained after pH shifting (p<0.05; FIGS. 8-10; FIG. 12), and precipitates were observed.

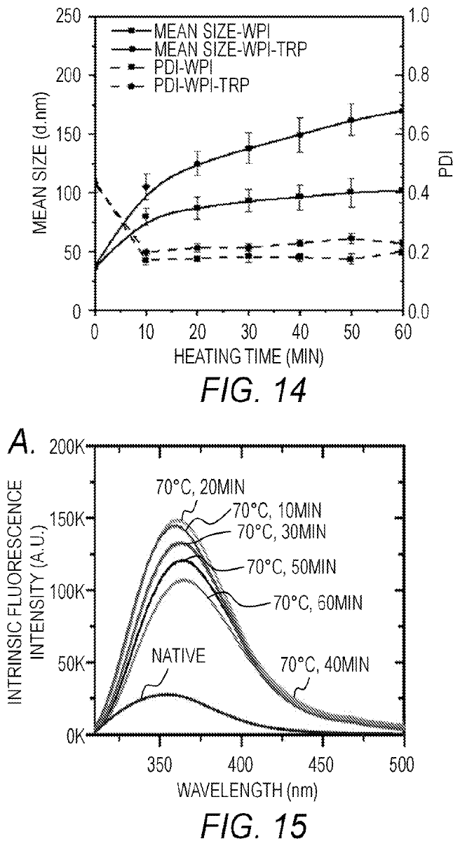

The difference in turbidity and particle size upon heating between WPI-Trp samples with pH shifting and WPI-Trp samples without pH shifting (FIGS. 8-13) may be explained by the modification of the WPI structure during pH shifting. Upon heating, WPI-Trp demonstrated a greater particle size and turbidity than WPI alone indicating that the WPI aggregation was enhanced in the presence of Trp (FIG. 14; FIGS. 8-13). Without wishing to be bound to any particular theory, this may be attributed to the increased interaction between the exposed hydrophobic residues on WPI with Trp.

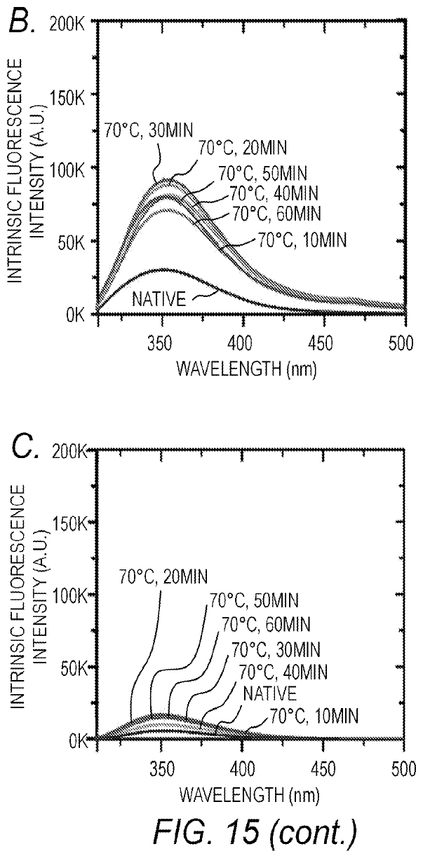

The fluorescence intensity of the WPI-Trp mixture reached its highest after 20 minutes of heat treatment (FIG. 15, Panel A). Heat treatment for longer than 20 minutes caused the fluorescence intensity to decrease and was accompanied by a slight red shift. This may be attributed to limited access of Trp to the interior of the protein due to strong protein-protein interaction resulting from extended heating times. Upon heating, the intrinsic fluorescence intensity of WPI samples that were pH-shifted increased during 30 minutes of heating due to protein aggregation and Trp residues in non-polar environments (FIG. 15, Panel B). After 30 minutes of heating, the fluorescence intensity began to decrease. The Trp fluorescence quenching could be due to the changes in the proximal amino acids and disulfides that occurred during aggregation. The intrinsic fluorescence intensity of Trp remained much lower than WPI and WPI-Trp samples (FIG. 15, Panel C). Based on the aggregation-induced emission, a strong quenching effect is believed to reduce the fluorescence emission when Trp molecules were freely dispersed in a hydrophilic environment.

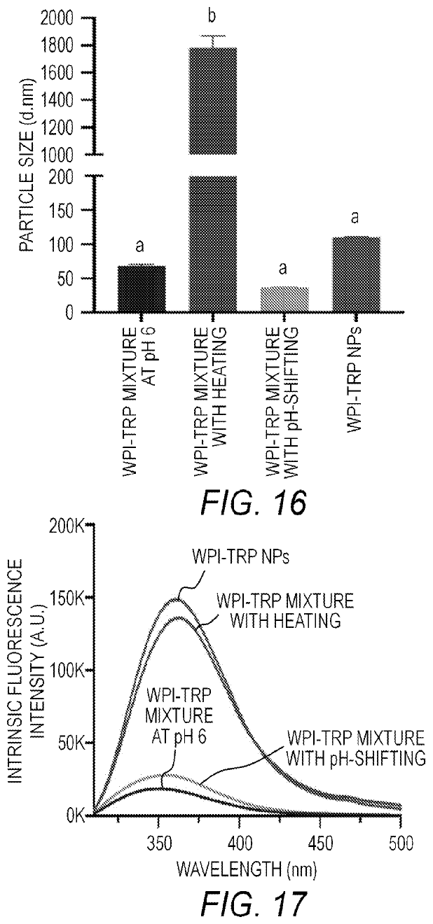

Native WPI-Trp at pH 6 had a particle size of 68.2±2.1 nm which decreased to 36.8±0.6 nm after pH shifting treatment and a slight increase in the intrinsic intensity accompanied by a red shift (FIG. 16). This is because the structure of whey protein in the molten-globule state was more flexible, with more hydrophobic residues exposed, which resulted in smaller particle sizes with greater Trp and thus higher fluorescence intensity.

WPI-Trp that was not pH-shifted, but heated, resulted in large particle sizes of 1784.0±85.4 nm where the heat-induced protein aggregation effect was substantial. While the pH-shifted and then heated WPI-Trp solutions showed a significantly smaller size at 110.1±0.8 nm (p<0.05), which indicated that pH shifting caused the WPI-Trp particles to be less thermally sensitive. Further, pH shifting combined with heating WPI-Trp had the highest fluorescence intensity, and the heating-only sample had a slightly lower peak (FIG. 17). It was believed that both heat and pH shifting treatment functionalized whey protein and contributed to Trp complexation.

### 3.4 Effect of Addition Methods and Concentration of HCl on WPI-Trp Nanoparticle Formation by pH Shifting and No Heating

The effect of an addition method and concentration of HCl on the WPI-Trp nanoparticles formation without heat treatment was investigated by either dropwise addition or direct mixing of a certain amount of 1-5M HCl solution into the basic solution of the WPI-mixture. This experiment demonstrated that, when adjusting the pH of a composition comprising WPI and Trp in a WPI:Trp ratio of 5:1 with a final total solids (WPI and Trp) concentration of 4.8% w/v from pH 11 to pH 6 using 1M HCl, clear differences were observed in the appearance of the suspensions formed by different addition methods. With dropwise addition of 1M HCl into WPI-Trp base solution after pH shifting from pH 11 to pH 6 without heat treatment, the suspension remained clear, indicating a lack of nanoparticle formation. However, direct mixing of the total volume of 1M HCl in a single addition to the mixture resulted in a colloidal dispersion instantaneously after pH shifting to from pH 11 to pH 6 without heat treatment, indicating nanoparticle formation without heat treatment. Further, when adjusting the pH from pH 11 to pH 6 using 5M HCl, both dropwise addition and direct mixing resulted in nanoparticle formation (colloidal dispersion) without heat treatment. This indicated the speed of adding HCl and/or the acid concentration significantly influenced the formation of the WPI-Trp nanoparticles. Therefore, the protocol for WPI-Trp nanoparticles formation could be varied by the addition methods and concentration of HCl during pH shifting. While not wishing to be bound to any particular theory, heat treatment may induce intermolecular association and allow for nanoparticles when adjusting pH from pH 11 to pH 6 by slowly adding HCl with low concentration. In addition, while not wishing to be bound to any particular theory, for the direct mixing method, the fast reaction may have induced molecular self-assembly nanoparticle formation and therefore heat treatment was not necessary in this case for WPI. This unique observation was not found for alpha-lactalbumin.

### 3.5 WPI-Trp Nanoparticle Size Distribution

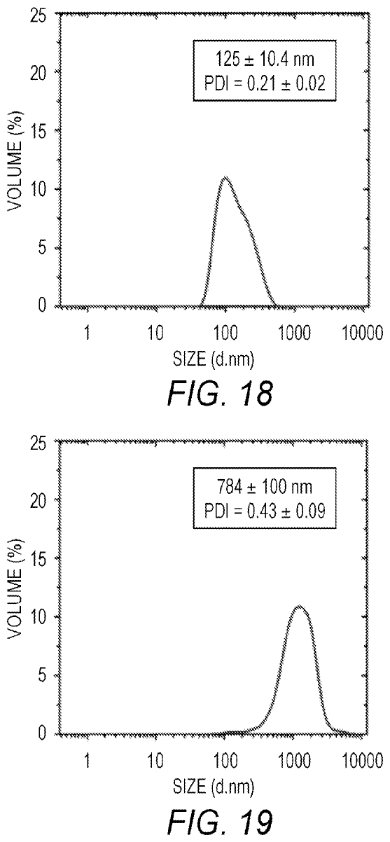

WPI-Trp nanoparticles were prepared with pH shifting from 11 to 6 followed by heat treatment at 70° C. for 20 minutes. As shown in FIG. 18, as compared to samples without pH shifting under the same conditions (shown in FIG. 19), the WPI-Trp nanoparticles formed with pH shifting had a particle size of about 125 nm with a narrow particle size distribution. The sample that underwent pH shifting also exhibited a less turbid suspension as compared to the sample that did not undergo pH shifting.

### 3.6 Heat Induced Interactions of WPI Nanoparticles in Skim Milk Dispersions

Dispersions containing Non-Fat Dry Milk Powder (NFDM) alone, NFDM and Whey Protein Isolate (WPI), NFDM and WPI nanoparticles, and NFDM and WPI-Tryptophan (Trp) nanoparticles were prepared by reconstituting the powders in deionized water to attain a final protein content of 4% w/w and a casein-to-protein ratio of 80:20 for dispersions containing NFDM alone and 60:40 for dispersions containing NFDM and WPI, NFDM and WPI nanoparticles, and NFDM and WPI-Trp nanoparticles. The powders were mixed for 30 min at 30° C. The composition of the dispersions is given in Table 1.

After overnight refrigerated storage, the dispersions were heated in a water bath to 30° C. followed by heating at 90° C. for 2 minutes. The dispersions were then cooled to room temperature immediately in an ice bath. The particle size of the dispersions were measured after the heat treatment to ascertain any heat-induced casein-whey protein interactions.

As shown in Table 1, the particle size of NFDM dispersions slightly decreased after heating. However, the particle size of NEDM/WPI dispersions (60:40 casein-to-whey protein dispersions) increased from about 310 nm to about 410 nm upon heating, which, while not wishing to be bound to any particular theory, is believed to be due to the heat-induced aggregation of casein-whey proteins or whey protein-whey protein interactions. The particle size of dispersions containing NFDM/WPINP or NFDM/WPITrpNP did not increase significantly after heating. These results demonstrate that the WPI nanoparticles and WPI-Trp nanoparticles that were prepared using a pH shifting and heating method can be used to produce functional WPI that does not interact on heating. These nanoparticles can be beneficial in applications such as dairy products where heat-induced interactions between proteins must be limited primarily to prevent heat-induced textural changes. For instance, the WPI nanoparticles ingredient can be used in high-protein yogurt formulations where interactions between casein and whey proteins must be minimized to manage viscosity and texture.

TABLE 1
Composition of NFDM and NFDM/WPI/WPINP/WPI-TrpNP dispersions
and their particle size before and after heating

| | | NFDM + | NFDM + WPI | NFDM + WPI-Trp |
|---|---|---|---|---|
| | NFDM/g | WPI/g | nanoparticles/g | nanoparticles/g |
| NFDM | 11.8 | 8.8 | 8.8 | 8.8 |
| WPI/WPNP/WPTrpNP | 0.0 | 1.1 | 1.1 | 1.3 |
| Water | 88.2 | 90.1 | 90.1 | 89.8 |
| Total | 100 | 100 | 100 | 100 |
| Casein:whey protein | 80:20 | 60:40 | 60:40 | 60:40 |
| Target protein content, % | 4 | 4 | 4 | 4 |
| Particle size before | 360 ± 4 | 318 ± 5 | 322 ± 3 | 325 ± 4 |
| heating (d · nm) | | | | |
| Particle size after heating | 349 ± 9 | 408 ± 21 | 341 ± 10 | 334 ± 3 |
| (d · nm) | | | | |

### 3.7 Characterization of WPI-Trp NPs Produced at Selected Conditions

Based upon the data, pH shifting from pH 11 to pH 6 followed by heating at 70° C. for 20 minutes was used to fabricate WPI-Trp nanoparticles. Under these conditions, the fluorescence intensity reached a maximum and the size of the nanoparticles was 110.1±0.8 nm with low PDI (0.20±0.02), which satisfied the requirements to be considered nanoparticles. Particles were obtained with pH shifting from pH 11 to pH 6 and/or heating at 70° C. for 20 minutes, optionally with freeze-drying as described further below.

### 3.7.1 SEM Imaging

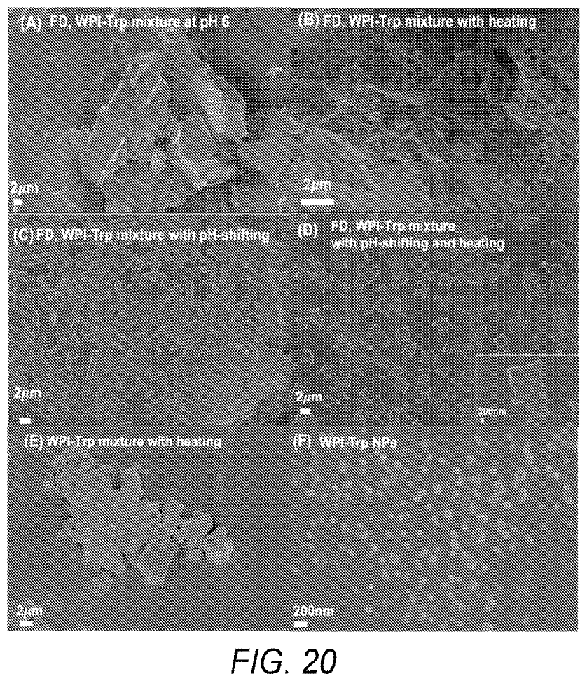

The microstructure of WPI-Trp with different processing treatments was analyzed using SEM (FIG. 20). The WPI-Trp sample at pH 6 after freeze-drying (FD) demonstrated large-scale flakes (FIG. 20, Panel A). Some samples were freeze dried after formation of the particles. Compared to the untreated sample, the WPI-Trp sample that was heated without pH shifting showed a greater level of aggregation and conjugation (FIG. 20, Panel B). The freeze-dried WPI-Trp sample with pH shifting (FIG. 20, Panel C) showed a rod-like shape. For freeze-dried WPI-Trp NPs samples obtained by pH shifting combined with heating (FIG. 20, Panel D), square-like aggregates were formed and evenly distributed with similar sizes, which suggested that the pH shifting samples self-aggregated to a more compact whey protein structure after heating (FIG. 20, Panel D). However, the size of freeze-dried WPI-Trp NPs was larger than the WPI-Trp NPs without freeze-drying (FIG. 20, Panel F), suggesting that freeze-drying may cause the further aggregation. The solution WPI-Trp NPs samples (before freeze-drying) with pH shifting combined with heating (FIG. 20, Panel F) showed evenly distributed nanoparticles with a spherical shape, while the solution samples prepared without pH shifting treatment and with heat treatment (FIG. 20, Panel E) showed dense and large aggregates. This result was consistent with the particle size distribution in which the heating alone WPI-Trp NP samples showed significantly larger particle sizes. Based on the difference in microstructure, we confirmed that the sample with pH shifting showed less thermal sensitivity compared to samples without pH shifting, thus supporting the formation of WPI-Trp NPs by the combination of pH shifting (pH 11 to pH 6) and heat treatment (70° C., 20 min).

### 3.7.2 Surface Hydrophobicity

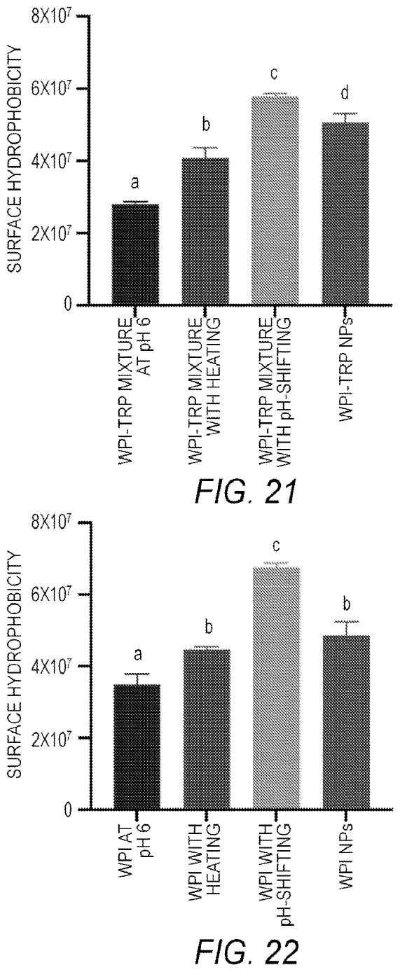

The surface hydrophobicity (H₀) of the protein indicates how many hydrophobic groups are exposed to the protein surface. The pH shifting treatment significantly increased the H₀ of WPI and WPI-Trp (FIGS. 21-22) which indicated that the initial interior hydrophobic groups of the native protein were exposed to the protein surface after pH shifting. This is also consistent with the red-shift of WPI, and the fluorescence spectra of WPI-Trp treated with pH shifting. WPI and WPI-Trp samples treated with heating also showed increased surface hydrophobicity (FIGS. 21-22). Without wishing to be bound to any particular theory, the red shift in the fluorescence spectra can be attributed to the heat treatment changing the spatial structure of the proteins and causing the proteins to unfold and expose hydrophobic groups on the protein surface. This increased surface hydrophobicity can enhance the non-covalent interactions between the WPI-Trp complexes.

The surface hydrophobicity of nanoparticles obtained by pH shifting combined with heating, however, was lower than the H₀ of samples that underwent pH shifting alone. This may be because, after pH shifting, the protein was in an unstable molten-globular state with certain hydrophobic groups exposed, showing a preference for hydrophobic interaction. Therefore, after heating, the exposed hydrophobic groups reburied again, reducing surface hydrophobicity. The aggregation induced by heating may be helpful to the complexation of WPI-Trp NPs. Overall, increased H₀ induced by pH shifting suggested unfolding and exposure of hydrophobic groups on WPI which facilitated hydrophobic interactions between WPI and Trp during the following heating process.

### 3.7.3 Free Sulfhydryl Group Content

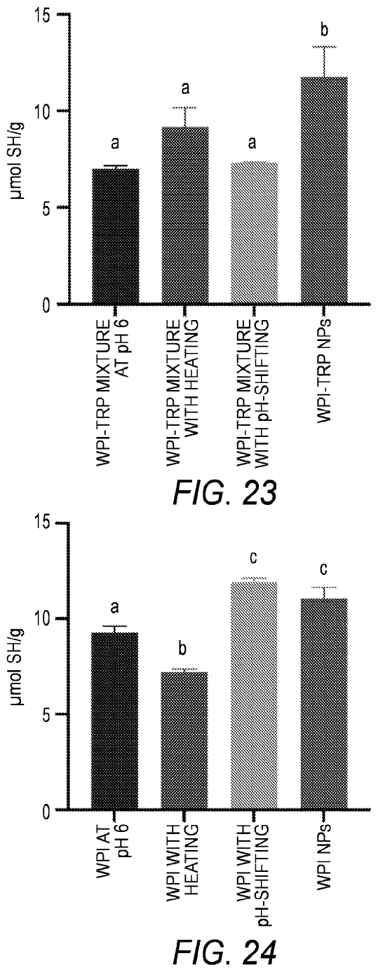

It is believed that the oxidation of and interchange between sulfhydryl (SH) groups and disulfide (S—S) bonds play an essential role in protein polymerization. Protein species in WPI, β-LG, α-La, and BSA, contain 2, 4, and 17 intramolecular S—S bonds, respectively. β-LG and BSA each contain one free thiol (—SH) group. Further, SH groups can accept radicals and be oxidized causing them to be converted to S—S bonds. After heat treatment of our WPI-Trp samples, the samples exhibited an increased SH group content (FIG. 23), which may be attributed to protein denaturation exposing free sulfhydryl groups. More specifically, when β-LG is heated, it separates into monomers which denature at high temperatures to expose a reactive sulfhydryl group, causing an increased SH group content. The increase was insignificant (p>0.05), though, because the oxidation of SH, or interchange SH/S—S reactions, was limited by the antioxidant property of Trp. By contrast, the total SH group content of WPI with heating was significantly lower than native WPI (p<0.05) (FIG. 24), which may be attributed to the interchange between the oxidation of the —SH or the S—S bond formation induced by heating. Oxidation of SH or conversion of SH into S—S bonding may facilitate the polymerization of WPI.

The SH group content of WPI significantly increased after pH shifting (p<0.05) (FIG. 24), which may be related to the unfolding of the protein, suggesting that SH groups that were originally buried in the protein were exposed or that disulfide bonds were broken in proteins. While SH content of WPI-Trp with pH shifting showed no significant difference compared with the untreated WPI-Trp mixture (p>0.05) (FIG. 23), this may also be related to the reduced oxidation of SH groups in the presence of Trp. WPI-Trp NPs obtained by pH shifting combined with heating showed the highest SH group content. Without wishing to be bound to any particular theory, this may be because pH shifting and heating synergistically enhanced the exposure of the SH group while suppressing the interchange between-SH groups and S—S bond formation due to the presence of Trp. The SH group content of WPI NPs obtained by pH shifting combined with heating was slightly lower than pH shifting WPI alone, which can also be attributed to the oxidation of reactive SH groups induced by heating. Overall, due to the presence of Trp, oxidation of SH groups to form S—S bonds might be limited during the formation of WPI-Trp NPs.

### 3.7.4 Circular Dichroism (CD)

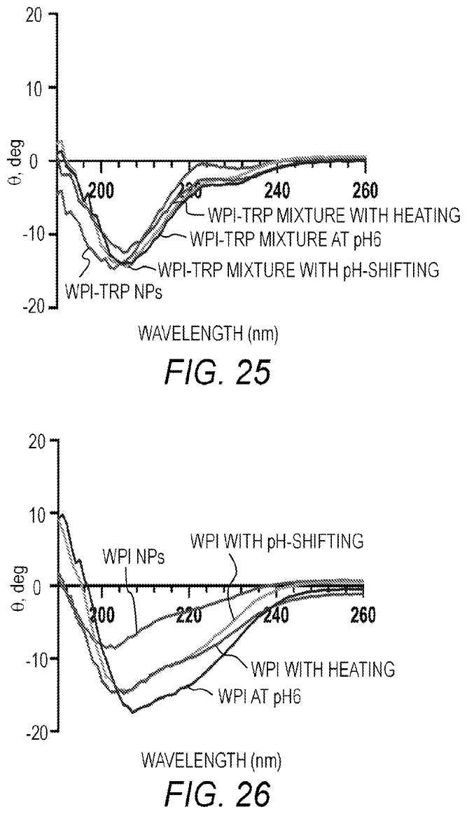

CD spectroscopy was used to identify the secondary structure of WPI and WPI-Trp and analyze the effect of pH shifting and heating on the secondary structure (FIGS. 25-26; Table 2). The untreated WPI exhibited a positive peak at 196 nm for the β-sheet structures and a negative peak at 208 nm for the α-helix structures. For WPI and WPI-Trp, both pH shifting treatment alone and heat treatment alone caused a blue-shift of a minimum of ellipticity (FIGS. 25-26) and thus a decrease in α-helix content (Table 2), indicating that the WPI protein was unfolded and short peptides were released. This loss of α-helix structure suggested that both heating and pH shifting can cause the protein to reach a molten-globule state with higher flexibility. Specifically, for WPI, both pH shifting alone and heating alone resulted in a significant decrease in α-helix content (p<0.05) and a significant increase in β-sheet and β-turn content (p<0.05). Conversely, WPI-Trp showed negligible changes in the content of α-helix, β-sheet, and β-turn content (p>0.05). Without wishing to be bound to any particular theory, this difference can be attributed to the interaction between WPI and Trp which promoted a more stable secondary structure. The pH shifting combined with heat treatment further decreased the α-helix content and caused a significant increase in random coil for both WPI-Trp NPs and WPI NPs (p<0.05), suggesting conversion of α-helix into random coil. This conversion demonstrated a more disordered structure of the modified whey protein with higher flexibility. Previous studies confirm that protein structures are less ordered with increasing content of random coil. This was consistent with a greater surface hydrophobicity of WPI-Trp NPs and WPI NPs with pH shifting combined with heating (FIGS. 21-22) because small hydrophobic patches were exposed after pH shifting and heating treatment. In conclusion, both heating and pH shifting may contribute to the disorder of WPI, which the molten-globule state of denatured protein may explain, and the presence of Trp may help stabilize the secondary structure of the protein.

TABLE 2
Secondary structure of WPI-Trp and WPI with different treatments.
Different letters indicate a significant difference (p < 0.05).

| | Area (%) | | | |
|---|---|---|---|---|
| | α-helix | β-sheet | β-turn | Random coil |
| WPI-Trp | | | | |
| Native | 17.70 ± 0.28^{a} | 28.25 ± 0.21^{a} | 23.65 ± 0.21^{a} | 30.45 ± 0.35^{a} |
| Heating | 15.50 ± 0.56^{b} | 29.4 ± 0.14^{b} | 23.85 ± 0.35^{a} | 31.3 ± 0.28^{b} |
| pH shifting | 16.35 ± 0.07^{a} | 28.3 ± 0.28^{a} | 23.6 ± 0.14^{a} | 31.7 ± 0.14^{b} |
| pH-shift and heating | 13.15 ± 0.91^{c} | 29.6 ± 0.71^{c} | 24.1 ± 0.71^{a} | 33.1 ± 0.99^{c} |
| WPI | | | | |
| Native | 22.1 ± 0.14^{a} | 24.85 ± 0.21^{a} | 22.50 ± 0.14^{a} | 30.60 ± 0.28^{a} |
| Heating | 17.45 ± 0.07^{b} | 26.55 ± 0.21^{b} | 24.20 ± 0.14^{a} | 31.80 ± 0.14^{b} |
| pH shifting | 18.85 ± 0.07^{c} | 26.75 ± 0.07^{b} | 23.90^{b} | 30.55 ± 0.07^{c} |
| pH-shift and heating | 9.35 ± 0.07^{d} | 34.00^{c} | 22.85 ± 0.21^{c} | 33.8 ± 0.28^{d} |

### 3.7.5 FTIR

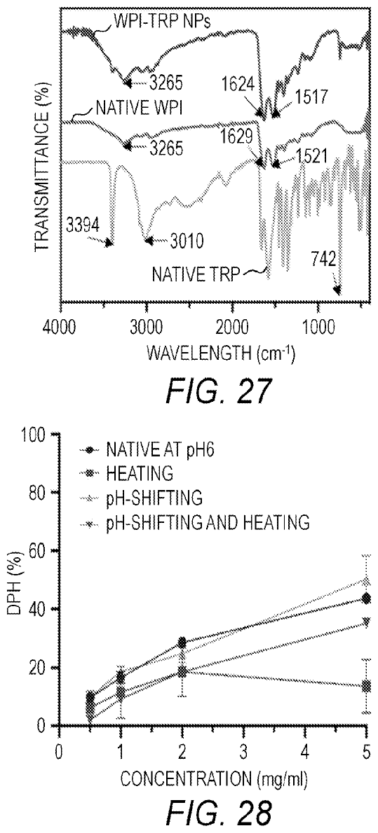

FTIR measurement was conducted to analyze the interactions between Trp and WPI (FIG. 27). The amide I band that appeared at 1600-1700 cm⁻¹ represented C═O stretching vibrations of the peptide bond. The amide II band was closely related to C—N stretching vibrations and bending vibration of N—H at 1500 cm⁻¹-1600 cm⁻¹ (Dai, L., Sun, C., Li, R., Mao, L., Liu, F., & Gao, Y. (2017). Structural characterization, formation mechanism and stability of curcumin in zein-lecithin composite nanoparticles fabricated by antisolvent co-precipitation. Food Chemistry, 237, 1163-1171; Zhong, M., Sun, Y., Sun, Y., Fang, L., Wang, Q., Qi, B., & Li, Y. (2022). Soy lipophilic protein self-assembled by pH-shift combined with heat treatment: Structure, hydrophobic resveratrol encapsulation, emulsification, and digestion. Food Chemistry, 394, 133514). Typical amide I and amide II absorption bands of WPI are located at 1629 cm⁻¹, and 1521 cm⁻¹, respectively. Compared to WPI, the WPI-Trp nanoparticles bands shifted to lower wavenumbers: 1624 cm⁻¹ for amide I and 1517 cm⁻¹ for amide II. The alteration of amides I and II suggested that hydrophobic interactions between WPI and Trp might occur. The band in the 3200-3500 cm⁻¹ region was associated with the —OH stretching vibrations in protein and are indicative of hydrogen bonding. The spectrum of WPI-Trp NPs had a band at 3265 cm⁻¹. Although no significant shifting was observed compared with the WPI, the wider and sharper band between 3200-3500 cm⁻¹ of WPI-Trp NPs suggested the formation of hydrogen bonding. For Trp, the sharp band at 3394 cm-1, 3010 cm⁻¹, and 742 cm⁻¹ corresponded to the N—H stretching vibrations of the indole ring, aromatic C—H stretching vibrations, and C—H bending vibrations in the aromatic ring, respectively.

After complexation with WPI, the characteristic bands of Trp at 3394 cm⁻¹ and 3010 cm⁻¹ disappeared, and the intensity of the band at 742 cm⁻¹ decreased, indicating interactions between the indole ring and WPI, which was probably related to the hydrogen bonding and the hydrophobic interaction between the indole ring of Trp and WPI. No new bands were detected in the FTIR spectra of WPI-Trp NPs compared with the WPI, suggesting that the interactions between WPI and Trp were mainly non-covalent bonding.

### 3.7.6 DPPH and ABTS Radical Scavenging Activity of WPI-Trp NPs

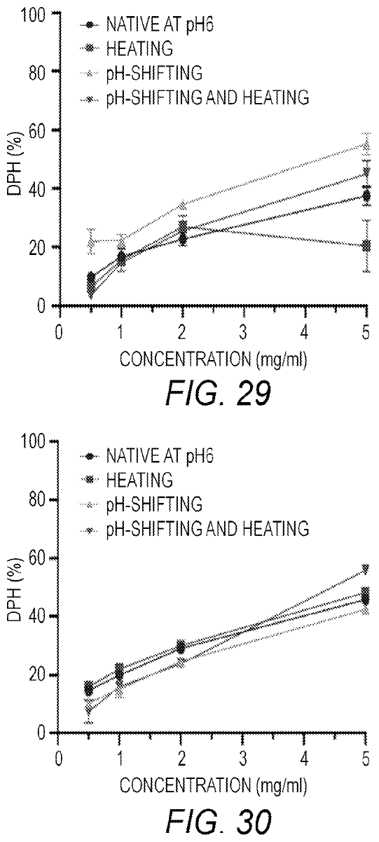

Relative ABTS and DPPH radical scavenging capacities of Trp, WPI, and WPI-Trp were measured to assess the impact of pH shifting and thermal treatments on antioxidant activity (FIG. 28-33). DPPH is an stable, oil-soluble free radical that can be used to measure radical scavenging activity. WPI-Trp after heat treatment alone demonstrated a lower DPPH radical scavenging capacity and showed the lowest DPPH radical scavenging capacity at 5 mg ml⁻¹ (FIG. 28). This may be because, after heat treatment, the exposed hydrophobic residues and hydrophobic interaction between WPI-Trp particles resulted in aggregation, resulting in low solubility in the DPPH solution. At 5 mg ml⁻¹, due to the low solubility of heated WPI-Trp and WPI samples in the DPPH solution, the DPPH radical scavenging capacity was the lowest. The DPPH radical scavenging capacity was not significantly influenced by the pH shifting treatment of WPI-Trp (p>0.05), while the DPPH scavenging capacity of WPI alone increased significantly (p<0.05) (FIG. 29). This may be because, after pH shifting treatment, interior amino acids of WPI were exposed, which enhanced the capacity to scavenge free DPPH radicals. For the WPI-Trp NPs obtained by pH shifting combined with heating, the DPPH scavenging capacity of WPI-Trp was reduced, although the encapsulation of Trp was greatest under these conditions (FIG. 28). This may be because the complexation of Trp with WPI stabilized the Trp and blocked access to the DPPH. The reducing effect of DPPH scavenging capacity has been shown in a previous study where the binding of whey protein concentrate with quercetin reduced the DPPH scavenging capacity because the hydrophobic cavity of whey protein concentrates isolated the quercetin from DPPH.

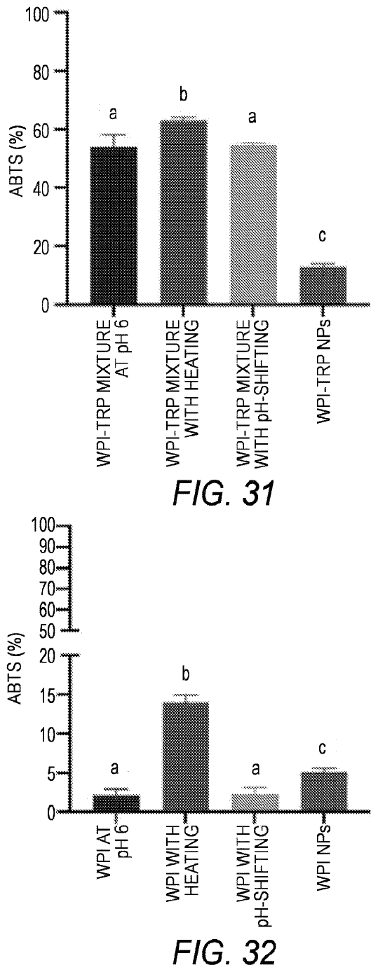

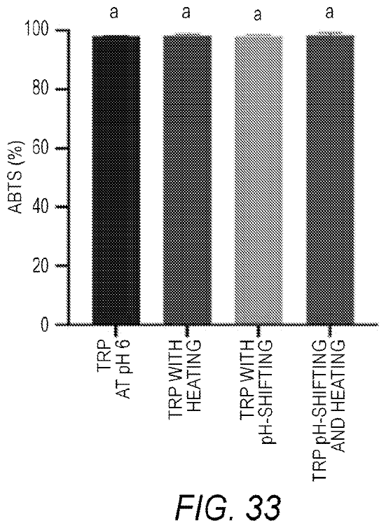

ABTS is a water-soluble radical and can be scavenged by antioxidants when the absorbance of ABTS is reduced. Due to the difference in solubility, the relative radical scavenging capacity of ABTS was higher than DPPH, and thus the ABTS radical scavenging capacity was demonstrated at a low sample concentration (0.05 mg mL⁻¹). Free Trp showed strong relative ABTS radical scavenging capacity and was not influenced by treatments (FIG. 33). The ABTS radical scavenging capacity of WPI-Trp with pH shifting combined with heating was significantly lower (p<0.05), suggesting the most significant content of free Trp was encapsulated (FIG. 31). The pH shifting combined with heating treatment was thus found to induce the strongest complexation of WPI-Trp nanoparticles. Nevertheless, the encapsulation of Trp caused lower antioxidant capacity because it was complexed within the protein and steric hindrance inhibited access to the radicals.

### 3.8 Molecular Docking

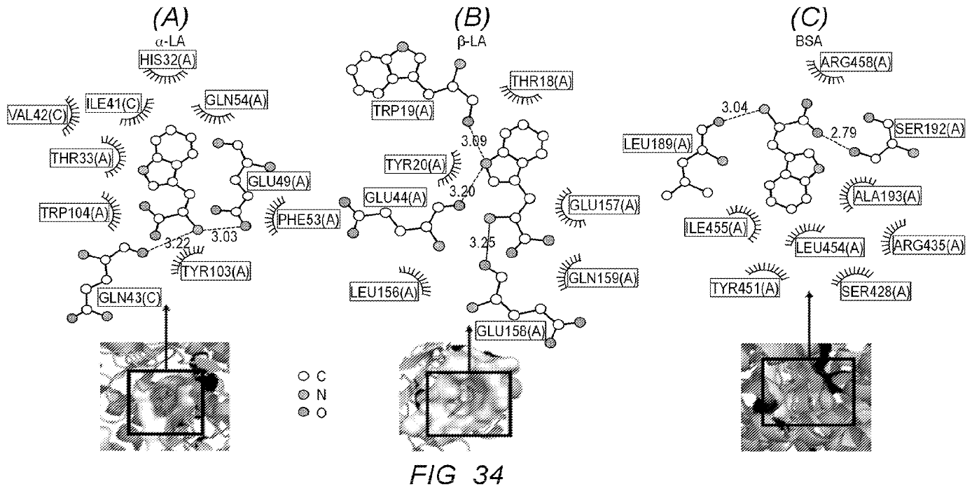

Molecular docking was used to investigate the interaction mechanism between Trp and WPI. The simulated nanocomplex of WPI-Trp and the three main protein species of WPI were investigated (α-LA, β-LG, and BSA), specifically docking for hydrogen bonding and hydrophobic interactions (FIG. 34). The amine group in the Trp can form two hydrogen bonds with Glu-49 and Gln-43 residues in α-LA, while other amino acids (Ile-41, Gln-54, His-32, Val-42, Thr-33, Trp-104, Tyr-103 and Phe-53) constituted a hydrophobic pocket to complex with Trp (FIG. 34, Panel A). In the case of β-LG, Trp-19 and Glu-44 constituted two hydrogen bonds with the amine group in the indole ring, and Glu-158 formed another hydrogen bond with the amino group of Trp (FIG. 34, Panel B). Amino acid residues of β-LG contributed to the hydrophobic interaction (Thr-18, Tyr-20, Glu-157, Gln-159 and Leu-156) with Trp. In terms of BSA, two hydrogen bonds were formed between the amino acid residues (Leu-189 and Ser-192), and Arg-458, Ile-455, Leu-454, Ala-193, Arg-435, Tyr-451, and Ser-428 were responsible for the hydrophobic interaction during the binding (FIG. 34, Panel C). In terms of the binding affinity (Table 3), α-LA was predicted to exhibit the highest binding score (−7.9 kcal/mol), followed by BSA (−6.6 kcal/mol) and β-LG (−5.8 kcal/mol), indicating that the α-LA showed the highest binding performance with the Trp. Thus, without wishing to be bound to any particular theory, our molecular docking results suggest that the formation of WPI-Trp complex was mainly driven by hydrophobic interactions and hydrogen bonding.

TABLE 3
Molecular docking results of different protein-Trp models

| Model | ΔG_{binding} (kJ/mol) |
|---|---|
| α-LA | −7.9 |
| β-LG | −5.8 |
| BSA | −6.6 |

### 3.9 Proposed Mechanism of the Formation of WPI-Trp NPs

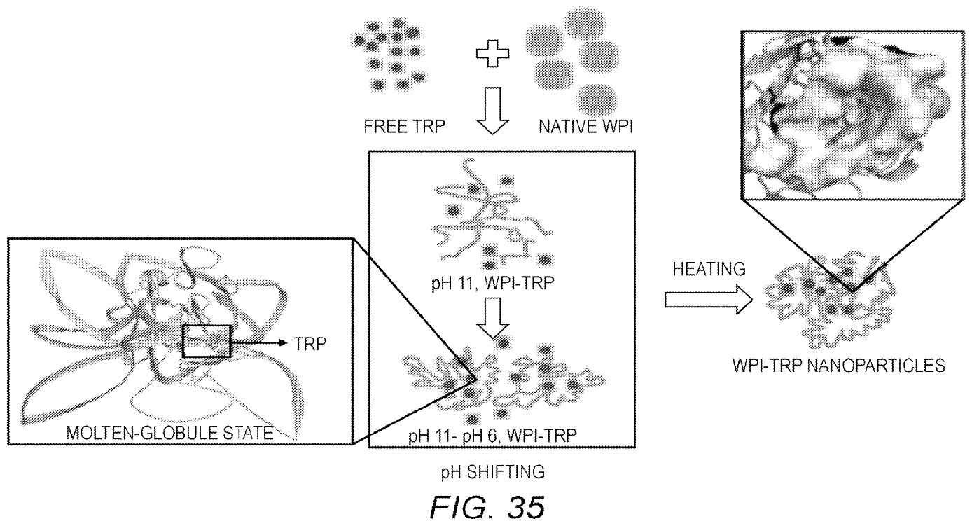

Without wishing to be bound to any particular theory, we propose the following mechanism for the formation of WPI-Trp NPs based on the data collected. The process may begin with unfolding and refolding of whey protein during pH shifting to place the protein into the molten globule state, where the interior hydrophobic residues are exposed (FIG. 35). Among these exposed residues, methionine and proline may bind with Trp. Free Trp may then be encapsulated into the hydrophobic cavity of the protein. Next, thermal treatment on the WPI-Trp after pH shifting may induce the aggregation. During the heating process, whey proteins may be denatured, and the interaction between exposed hydrophobic residues, exposure of reactive SH groups, and exchange reactions between SH and S—S may occur, all of which may contribute to the complexation of WPI-Trp NPs.

Without wishing to be bound to any particular theory, pH shifting treatment encourages the exposure of hydrophobic residues, including inner fluorophores (tyrosine, tryptophan, and phenylalanine), which can increase the binding sites for Trp. Without wishing to be bound to any particular theory, heat treatment encourages the aggregation of WPI, which can further encapsulate the Trp. Based on the aggregation-induced emission, encapsulation of added Trp was confirmed. Evaluation of the prepared particles demonstrated that pH shifting combined with heat treatment created WPI-Trp nanoparticles that were less thermally sensitive than particles produced through heating alone. Non-covalent interactions, including hydrogen bonding and hydrophobic interactions, were the main driving force for the complexation of WPI-Trp nanoparticles.

The foregoing is illustrative of the present invention, and is not to be construed as limiting thereof. The invention is defined by the following claims, with equivalents of the claims to be included therein.

1. A method of preparing a particle, the method comprising:
providing a protein in a composition;
adjusting the pH of the composition to a basic pH, then
adjusting the pH of the composition to an acidic pH; and
optionally, then heating the composition,
thereby forming the particle that comprises the protein.

2. The method of claim 1, further comprising, prior to adjusting the pH of the composition, adding an active agent to the composition, and wherein the particle comprises the protein and the active agent.

3. The method of claim 1, wherein the protein:
is a dairy protein, a plant protein, and/or an animal protein;
has about 100 amino acids to about 500 amino acids;
has a molecular weight of about 10 kDa to about 50 kDa; and/or
has an isoelectric point (pI) of about 4 to about 5.5.

4. The method of claim 1, wherein the protein is selected from the group consisting of whey protein isolate, alpha-lactalbumin, lysozyme, cytochrome c, apomyoglobin, staphylococcal nuclease, and any combination thereof.

5. The method of claim 2, wherein the active agent is selected from the group consisting of tryptophan, leucine, phenylalanine, cysteine, tyrosine, vitamin E, and any combination thereof.

6. The method of claim 1, wherein the composition is an aqueous composition comprising the protein.

7. The method of claim 2, wherein the composition is an aqueous composition comprising the protein and the active agent.

8. The method of claim 2, wherein the composition comprises the protein and the active agent in a weight ratio of about 2:1 to about 30:1 (protein:active agent).

9. The method of claim 1, wherein the composition has a total solids content in a range of about 1% w/v to about 10% w/v.

10. The method of claim 1, wherein adjusting the pH of the composition to the basic pH comprises adjusting the pH of the composition to a pH of about 8 to about 13.

11. The method of claim 1, wherein adjusting the pH of the composition to the acidic pH occurs about 15 minutes to about 40 minutes after the adjusting of the pH of the composition to the basic pH.

12. The method of claim 1, wherein adjusting the pH of the composition to the acidic pH comprises adjusting the pH of the composition to a pH of about 2 to about 6.5.

13. The method of claim 1, wherein the method comprises heating the composition for about 10 minutes to about 60 minutes.

14. The method of claim 1, wherein the method comprises heating the composition to a temperature in a range from about 50° C. to about 80° C.

15. The method of claim 1, wherein adjusting the pH of the composition to the basic pH comprises adding a base to the composition and/or wherein adjusting the pH of the composition to the acidic pH comprises adding an acid to the composition.

16. (canceled)

17. The method of claim 1, wherein the particle has a particle size distribution in a range of about 50 nm to about 700 nm with a polydispersity index of less than about 0.5 and/or wherein the particle has a mean particle size of about 50 nm to about 200 nm.

18. (canceled)

19. The method of claim 2, wherein the active agent is present in the particle in an amount of about 25% or less by weight of the particle and/or the protein is present in the particle in an amount of about 75% to about 100% by weight of the particle.

20. A particle comprising:
a protein; and
optionally an active agent,
wherein, when the active agent is present in the particle, the active agent is present within the protein.

21-49. (canceled)

50. A composition comprising a carrier and the particle prepared according to the method of claim 1.

51. (canceled)

52. An article comprising the particle prepared according to the method of claim 1, wherein the article is a food product, nutritional supplement, therapeutic drink, and/or cosmetic.

53. (canceled)


SEQUENCE LISTING (3 sequences)

SEQ ID NO: 1 - protein, 123 aa, Bos taurus
EQLTKCEVFRELKDLKGYGGVSLPEWVCTTFHTSGYDTQAIVQNNDSTEYGLFQINNKIWCKDDQNPHSSNICNISCDKFLDDDLTDDIMCVKKILDKVGINYWLAHKALCSEKLDQWLCEKL

SEQ ID NO: 2 - protein, 162 aa, Bos taurus
LIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 3 - protein, 708 aa, Bos taurus
MKLFVPALLSLGALGLCLAAPRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR